(19) United States
(12) Patent Application Publication
(10) Pub. No.: US 2015/0297885 A1
(43) Pub. Date: Oct. 22, 2015

(54) Implantable Electrical Stimulation Leads

(71) Applicant: EndoStim, Inc., St. Louis, MO (US)

(72) Inventors: Paul V. Goode, Round Rock, TX (US); Ofer Glasberg, Zichron Ya'akov (IL); Virender K. Sharma, Paradise Valley, AZ (US); Shai Policker, Tenafly, NJ (US); Alejandro Nieponice, Buenos Aires (AR)

(21) Appl. No.: 14/753,402

(22) Filed: Jun. 29, 2015

Related U.S. Application Data
(63) Continuation-in-part of application No. 14/191,085, filed on Feb. 26, 2014.
(60) Provisional application No. 62/020,652, filed on Jul. 3, 2014.
(60) Provisional application No. 61/769,732, filed on Feb. 26, 2013.

(51) Int. Cl. A61N 1/05; A61B 17/04; A61B 17/00

(52) U.S. Cl. CPC A61N 1/0509; A61B 17/00234; A61B 17/0469; A61B 2017/00296; A61B 2017/00818

(57) ABSTRACT

Implantable electrical stimulation leads for the treatment of biological conditions include a lead body with an electrical connector at one end and multiple in-line electrodes at the other end. The lead body has a length ranging from 350 mm to 630 mm to allow for implantation from an incision site further removed from the final positioning site of the electrodes. One lead has a suture loop extending from the most distal electrode for pulling the lead through the working channel of an endoscope. Another lead has a length of suture with a free end attached to the most distal electrode. Yet another lead has a length of suture attached to the most distal electrode at one end and a needle at the other end. The needle has a curve designed to facilitate maneuvering in confined anatomy. The lead having the needle is designed to be implanted laparoscopically.

### CROSS-REFERENCE

The present application relies on U.S. Provisional Patent Application No. 62/020,652, entitled “Implantable Electrical Stimulation Leads” and filed on Jul. 3, 2014, for priority.

The present application is also a continuation-in-part application of U.S. patent application Ser. No. 14/191,085, entitled “Implantable Electrical Stimulation Leads” and filed on Feb. 26, 2014, which relies on U.S. Provisional Patent Application No. 61/769,732, of the same title and filed on Feb. 26, 2013, for priority. All of the aforementioned applications are herein incorporated by reference in their entirety.

The present specification is related to U.S. patent application Ser. No. 13/602,184, entitled “Endoscopic Lead Implantation Method”, filed on Sep. 2, 2012, and assigned to the applicant of the present invention, which is herein incorporated by reference in its entirety.

### FIELD

The present specification relates generally to implantable leads used in the electrical stimulation of human tissues. More particularly, the present specification relates to implantable electrical stimulation leads useful in the stimulation of anatomical structures proximate the gastroesophageal junction.

### BACKGROUND

Electrical stimulation of nerves and surrounding tissue is used to treat a variety of conditions. For example, electrical stimulation can be used to restore partial function to limbs or organs following traumatic injury. Electrical stimulation can also be used to reduce pain. Specifically, electrical stimulation can be used to treat disorders associated with the gastrointestinal (GI) system, such as, obesity and gastroesophageal reflux disease (GERD).

Obesity is a common condition and a major public health problem in developed nations including the United States of America. As of 2009, more than two thirds of American adults, approximately 127 million people, were either overweight or obese. Data suggest that 300,000 Americans die prematurely from obesity-related complications each year. Many children in the United States are also either overweight or obese. Hence, the overall number of overweight Americans is expected to rise in the future. It has been estimated that obesity costs the United States approximately $100 billion annually in direct and indirect health care expenses and in lost productivity. This trend is also apparent in many other developed countries.

For adults, the body mass index (BMI) is used to determine if one is overweight or obese. A person's BMI is calculated by multiplying body weight in pounds by 703 and then dividing the total by height in inches squared. A person's BMI is expressed as kilograms per meter squared. An adult is considered overweight if his or her BMI is between 25 and 30 kg/m2. Obesity is defined as possessing a BMI between 30 and 40 kg/m2. A BMI greater than 30 kg/m² is associated with significant co-morbidities. Morbid obesity is defined as possessing either a body weight more than 100 pounds greater than ideal or a body mass index (BMI) greater than 40 kg/m². Approximately 5% of the U.S. population meets at least one of the criteria for morbid obesity. Morbid obesity is associated with many diseases and disorders including, for example: diabetes; hypertension; heart attacks; strokes; dyslipidemia; sleep apnea; pickwickian syndrome; asthma; lower back and disc disease; weight-bearing osteoarthritis of the hips, knees, ankles and feet; thrombophlebitis and pulmonary emboli; intertriginous dermatitis; urinary stress incontinence; gastroesophageal reflux disease (GERD); gallstones; and, sclerosis and carcinoma of the liver. In women, infertility, cancer of the uterus, and cancer of the breast are also associated with morbid obesity. Taken together, the diseases associated with morbid obesity markedly reduce the odds of attaining an average lifespan. The sequelae raise annual mortality in affected people by a factor of 10 or more.

Gastro-esophageal reflux disease (GERD) is another common health problem and is expensive to manage in both primary and secondary care settings. This condition results from exposure of esophageal mucosa to gastric acid as the acid refluxes from the stomach into the esophagus. The acid damages the esophageal mucosa resulting in heartburn, ulcers, bleeding, and scarring, and long term complications such as Barrett's esophagus (pre-cancerous esophageal lining) and adeno-cancer of the esophagus.

Gastric electrical stimulation (GES) is aimed at treating both obesity and GERD. GES employs an implantable, pacemaker-like device to deliver low-level electrical stimulation to the gastrointestinal tract. For obesity, GES operates by disrupting the motility cycle and/or stimulating the enteric nervous system, thereby increasing the duration of satiety experienced by the patient. The procedure involves the surgeon suturing electrical leads to the outer lining of the stomach wall. The leads are then connected to the device, which is implanted just under the skin in the abdomen. Using an external programmer that communicates with the device, the surgeon establishes the level of electrical stimulation appropriate for the patient. The Abiliti® implantable gastric stimulation device, manufactured by IntraPace, is currently available in Europe for treatment of obesity.

In another example, Medtronic offers for sale and use the Enterra™ Therapy, which is indicated for the treatment of chronic nausea and vomiting associated with gastroparesis when conventional drug therapies are not effective. The Enterra™ Therapy uses mild electrical pulses to stimulate the stomach. According to Medtronic, this electrical stimulation helps control the symptoms associated with gastroparesis, including nausea and vomiting.

Electrical stimulation has also been suggested for use in the treatment of GERD, wherein the stimulation is supplied to the lower esophageal sphincter (LES). For example, in U.S. Pat. No. 6,901,295, assigned to Endostim, Inc., “A method and apparatus for electrical stimulation of the lower esophageal sphincter (LES) is provided. Electrode sets are placed in the esophagus in an arrangement that induce contractions of the LES by electrical stimulation of the surrounding tissue and nerves. The electrical stimulus is applied by a pulse generator for periods of varying duration and varying frequency so as to produce the desired contractions. The treatment may be short-term or may continue throughout the life of the patient in order to achieve the desired therapeutic effect. The stimulating electrode sets can be used either alone or in conjunction with electrodes that sense esophageal peristalsis. The electrode sets can be placed endoscopically, surgically or radiologically.” The referenced invention relies on sensing certain physiological changes in the esophagus, such as changes in esophageal pH, to detect acid reflux. Once a change in esophageal pH is recognized, the system generates an electrical stimulation in an attempt to instantaneously close the LES and abort the episode of acid reflux. U.S. Pat. No. 6,901,295 is hereby incorporated by reference in its entirety.

The leads used in electrical stimulation of gastrointestinal tissues traditionally comprise elongated or coiled, insulated wires or cables having a means for attachment to an electrical pulse generator at one end and one or more exposed electrodes at the other end. The leads are typically anchored in place such that the electrodes are positioned and remain proximate the target nerve or tissues. Anchoring is often accomplished by suturing the electrode containing ends of the leads proximal to the electrodes and into the surrounding tissue. Traditional leads often comprise a needle attached to a length of suture nylon at the distal end of each branch of the lead. A butterfly shaped anchoring element is positioned on each branch just proximal to each electrode. The needle and suture nylon are used to create a pathway for the electrode to be inserted into the tissue, with the needle and most of the suture being removed thereafter. The remaining suture is used as a tether onto which at least one clip (e.g., titanium clip) is used to provide a distal stop thus preventing the electrode from backing out until sufficient fibrosis is formed.

While current electrical leads are effective in transmitting electrical stimulation to target nerves and tissues, they are not without their drawbacks. For example, the overall length of current leads limits the implantation site of the stimulator to which they connect. A lead that is intended to have its electrodes positioned proximate the gastroesophageal junction is often implanted through the abdominal wall via laparoscopy, but requiring the stimulator and its unsightly scar at the patient's exposed abdomen. Therefore, what is needed is a lead having an increased overall length to permit stimulator implantation at points further from the therapy site, whereby the scar could be covered by most clothing apparel (e.g., male and female swimsuits) or the implant access could be through the umbilicus.

In addition, with regard to bipolar leads, the monopolar branches that extend beyond the bifurcation point are often too long. Lengthy monopolar branches can become entangled in surrounding tissues, leading to dislodgment of anchored leads and stricture formation. Therefore, what is needed is a bipolar lead having shortened monopolar branches. Further, traditional leads are often pulled backward to facilitate anchoring, causing the proximal 2 to 3 mm of conductive material to become exposed. Exposed conductive material can result in inadvertent electrical stimulation of non-target tissues as well as less stimulation current reaching the target tissues. Therefore, what is also needed is a lead having additional insulation closer to the electrodes.

Traditional leads also include electrodes that are too large for certain applications, including stimulation of the gastroesophageal junction. Oversized electrodes can also result in inadvertent electrical stimulation of non-target tissues. Therefore, what is needed is a lead having smaller sized electrodes. In addition, the space in which to work surrounding the gastroesophageal junction (GEJ) is relatively confined compared to other spaces, such as, around the body of the stomach. Traditional leads having long suture nylons tempt the surgeon to use the same needle and suture for anchoring the lead proximal to the electrode; however, this suture material is chosen for applying distal clips and not anchoring the leads. Therefore, what is also needed is a lead having shorter suture nylons on each branch such that this needle and suture is not long enough to be used for anchoring the leads proximal to the electrode. Having shorter suture nylons also reduces the number of pulling maneuvers required in order to bring the electrode(s) into final position. Traditional leads often include a curved needle for anchoring. The degree of curvature of the needle is often not sufficient when considering the adjacent tissues, resulting in injury to the tissue. What is needed is a needle curvature which will allow the user to significantly bury the electrode within the target tissue while also making the needle easily retrievable from the tissue exit site without puncturing or scraping nearby tissues.

Therefore, what is needed specifically for GEJ implantation is a lead having a needle with a degree of curvature specific to the target and surrounding tissue. Some traditional leads include an additional suture sleeve over the lead body to prevent damage to surrounding tissues during implantation. However, this sleeve tends to attract much fibrosis. Therefore, what is also needed is a lead having no additional anchoring sleeve.

Traditional leads are often implanted laparoscopically via an incision site on the abdomen. The incision typically leaves several visible scars and use of anchoring needles usually results in some trauma to the internal tissues. Applying suture anchors through an endoscope are difficult, specifically in the confined space of the GEJ or in a small endoscopic tunnel. Therefore, there is also a need for an electrical lead that can be implanted using an endoscope and can be anchored to surrounding tissues without using needles and sutures.

### SUMMARY

The present specification discloses an in-line implantable electrical lead for use in the stimulation of biological tissues, said lead comprising: an insulated, flexible, elongate lead body having a proximal end and a distal end; a connector attached to and in electrical communication with said proximal end of said lead body; a plurality of electrodes comprising at least a most proximal electrode and a most distal electrode, said electrodes being arranged in-line and spaced a predetermined distance apart from one another, wherein said most proximal electrode is attached to said distal end of said lead body; at least one conductor positioned between and extending through each of said plurality of electrodes, thereby connecting each of said plurality of electrodes; and, a suture extending distally from said most distal electrode; wherein a first length extending from a proximal end of said connector to a distal end of said most proximal electrode is in a range of 450 to 550 mm and a second length of said conductor is in a range of 1 to 50 mm.

The plurality of electrodes may be equal to two, four, and eight.

Each of said plurality of electrodes may have a length in a range of 1 to 25 mm and a width in a range of 0.10 to 1.50 mm.

The lead body may be comprised of a plurality of coils or cables.

The width of said lead body may be in a range of 0.20 to 2.00 mm.

The conductor may be comprised of a plurality of conductors.

The lead may comprise more than two electrodes and two or more conductors. Each conductor may have the same or different lengths or some conductors may have the same length while other conductors have different lengths.

Optionally, the implantable electrical lead further comprises a suture loop and a suture tail formed from said suture extending distally from said second electrode. The diameter of said suture loop about its widest point may be in a range of 1 to 20 mm. A third length extending from a distal end of said second electrode to a knot forming said loop may be in a range of 1 to 20 mm and a fourth length of said suture tail may be in a range of 100 to 500 mm. The diameter of said suture loop may be fixed. The diameter of said suture loop may be adjustable by pulling on a portion of said suture, suture loop, or suture tail.

Optionally, the implantable electrical lead further comprises a needle attached to a distal end of said suture. The needle may be within a range of a ¼ to ⅜ of a circle curve needle with a length ranging from 13 to 28 mm and may include a base having a diameter in a range of 0.58 mm to 0.88 mm.

Optionally, the needle comprises a straight proximal portion having a first length within a range of 8 mm to 16 mm, a curved distal portion having a second length within a range of 4 mm to 10 mm, and an opening at a proximal end of said straight proximal portion configured to fixedly receive a length of suture and extending at least 1.6 mm within said straight proximal portion, further wherein a tapered point at a distal end of said curved distal portion is offset from an axis of said straight proximal portion by a distance within a range of 1 mm to 5 mm.

Optionally, the implantable electrical lead further comprises a sleeve covering a proximal portion of said lead body and a distal portion of said connector. Optionally, the implantable electrical lead further comprises a retention ring positioned proximal to said sleeve and securing said sleeve in place.

The present specification also discloses an in-line implantable electrical lead for use in the stimulation of biological tissues, said lead comprising: an insulated, flexible, elongate lead body having a proximal end and a distal end; a connector attached to and in electrical communication with said proximal end of said lead body; a first electrode attached to said distal end of said lead body; a second electrode attached to said first electrode by a connecting conducting cable, said second electrode being in-line with and spaced distally apart from said first electrode; and, a suture extending distally from said second electrode; wherein a first length extending from a proximal end of said connector to a distal end of said first electrode is in a range of 450 to 550 mm and a second length of said connecting conducting cable is in a range of 1 to 50 mm.

The present specification also discloses a method of endoscopically implanting an electrical stimulation lead having a connector, a lead body, a first electrode, a second electrode in-line with said first electrode, and a suture extending distally from said second electrode, said method comprising the steps of: stitching said suture at least once through the muscularis of a lower esophageal sphincter (LES); tying a distal end of said suture to a proximal end of said suture; pulling on a distal end of said suture to pull said lead body into an esophagus; pushing said lead body into a stomach using graspers; pulling on said distal end of said suture to thread electrodes into stitch path; suturing at least one additional suture and T-tag through a suture loop created with said suture of said lead; removing excess suture from said lead; creating a gastric port using a percutaneous endoscopic gastrostomy (PEG) procedure; and, delivering said lead through said gastric port.

Optionally, the lead further includes a loop formed from said suture and said steps of pulling on said distal end of said suture comprise pulling on said loop.

The present specification also discloses a method of implanting an electrical stimulation lead having a connector and a plurality of in-line electrodes into a patient, said method comprising the steps of: inserting a distal end of an endoscope into a natural orifice of said patient; creating a tunnel under a gastric mucosa starting 5 cm to 10 cm proximal to the gastroesophageal junction (GEJ); tunneling 5 cm to 10 cm distal to the GEJ on an anterior gastric wall; creating a gastropexy to bring the anterior gastric wall to an abdominal wall; introducing a needle through the skin into the mucosal tunnel while under surveillance using the endoscope and/or ultrasound to guide the needle to the correct location; introducing a peel-away introducer over the needle into the mucosal tunnel under guidance from the endoscope; removing the needle; inserting the electrical stimulation lead into the introducer and feeding it into the mucosal tunnel under guidance from the endoscope; grasping a suture portion of the implantable electrical lead using endoscopic graspers; pulling the electrical stimulation lead such that the electrodes are positioned in or proximate the lower esophageal sphincter (LES); removing the introducer; closing an opening of the musical tunnel proximal to the LES; connecting the electrical stimulation lead connector into an implantable pulse generator; placing the implantable pulse generator in a subcutaneous pocket; and programming the implantable pulse generator to deliver therapy.

Optionally, the electrical stimulation lead is anchored to the muscularis of the LES by any conventional suturing mechanism.

Optionally, the electrical stimulation lead is anchored to the muscularis of the LES by using sutures which contain micro-barb structures.

Optionally, the electrical stimulation lead is anchored to the muscularis of the LES by employing a barb-like element which anchors itself when the lead is pulled.

Optionally, the electrical stimulation lead is anchored to the muscularis of the LES by use of a biomaterial which promotes tissue in-growth including any one or combination of porous silicone and tissue scaffolds.

The present specification also discloses an implantable electrical lead for use in the stimulation of biological tissues, said lead comprising: an elongate lead body having a proximal end and a distal end, said lead body comprising an electrically conductive inner coil, an electrically conductive outer coil, a first insulating sheath covering said inner coil, and a second insulating sheath covering said outer coil wherein said lead body has a length within a range of 390 mm to 590 mm; a connector attached to and in electrical communication with said proximal end of said lead body; a first elongate branch having a proximal end and a distal end, said first elongate branch comprising said inner coil and said first insulating sheath covering said inner coil and not comprising said outer coil and said second insulating sheath, wherein said first branch has a length within a range of 20 mm to 150 mm; a second elongate branch having a proximal end and a distal end, said second elongate branch comprising said outer coil and said second insulating sheath covering said outer coil and not comprising said inner coil and said first insulating sheath, wherein said proximal end of said first branch and said proximal end of said second branch join to form said distal end of said lead body, wherein said second branch has a length within a range of 20 mm to 150 mm; a first anchoring element and a first electrode attached to said first branch and positioned proximate said distal end of said first branch; and, a second anchoring element and a second electrode attached to said second branch and positioned proximate said distal end of said second branch.

Optionally, the implantable electrical lead further comprises a first length of suturing material and a second length of suturing material, each having a proximal end and a distal end, wherein said proximal end of said first length of said suturing material is attached to said distal end of said first branch and said proximal end of said second length of said suturing material is attached to said distal end of said second branch. In various embodiments, the first and second lengths of suturing material are each in a range of 40 to 80 mm. In one embodiment, the implantable electrical lead further comprises a first needle attached to said distal end of said first length of suturing material and a second needle attached to said distal end of said second length of suturing material, wherein said first needle and said first length of suturing material are used to suture said first anchoring element to a biological tissue and said second needle and said second length of suturing material are used to suture said second anchoring element to a biological tissue. In various embodiments, the first and second needles are each within a range of ¼ to ⅜ of a circle curve needles with a length ranging from 13 to 28 mm and include a base having a diameter in a range of 0.58 mm to 0.88 mm. Optionally, the first and second needles comprises a straight proximal portion having a first length within a range of 8 mm to 16 mm, a curved distal portion having a second length within a range of 4 mm to 10 mm, and an opening at a proximal end of said straight proximal portion configured to fixedly receive a length of suture and extending at least 1.6 mm within said straight proximal portion, further wherein a tapered point at a distal end of said curved distal portion is offset from an axis of said straight proximal portion by a distance within a range of 1 mm to 5 mm.

Optionally, wherein a distal end of said outer coil is positioned at said distal end of said lead body, said lead further comprises an additional electrically conductive coil having a proximal end and a distal end and comprising said second branch, wherein said proximal end of said additional coil is attached to said distal end of said outer coil and said second anchoring element and said second electrode are attached to and positioned proximate said distal end of said additional coil and said second insulating sheath extends over said additional coil.

Optionally, the implantable electrical lead further comprises a sleeve covering the distal end of said lead body and the proximal ends of said first branch and said second branch.

Optionally, the implantable electrical lead further comprises a marking element on said first branch to serve as a visual indicator.

Optionally, said first insulating sheath extends over a proximal portion of said first electrode and said second insulating sheath extends over a proximal portion of said second electrode such that, after said lead is implanted, said insulating sheaths are pulled partially in a proximal direction to expose said proximal portions of said electrodes. In various embodiments, the first and second insulating sheaths extend in a range of 1 to 10 mm over said first and second electrodes. In various embodiments, after said lead is implanted, a total exposed length of said electrodes is in a range of 1 to 10 mm.

The present specification also discloses a lead delivery catheter to be used with an endoscope or a laparoscope and for implanting the electrical stimulation lead described above in the body of a patient, said catheter comprising: a catheter body having a proximal end, a distal end, and a lumen within; an inflatable balloon attached to said distal end of said catheter body; and, a grasping mechanism attached to said distal end of said catheter body for grasping said lead.

Optionally, the catheter further comprises a light source providing illumination at its distal end.

Optionally, the catheter further comprises a camera at its distal end.

Optionally, the catheter further comprises a bipolar electrocautery electrode at its distal end. In one embodiment, the bipolar electrocautery electrode is incorporated into said grasping mechanism.

The present specification also discloses an implantable electrical lead for use in the stimulation of biological tissues, said lead comprising: a Y shaped structure comprising a central portion, having a proximal end and a distal end, a first prong, and a second prong, each prong having a proximal end and a distal end, wherein said proximal ends of said first and second prongs join together to form said distal end of said central portion, further wherein: said central portion comprises an electrically conductive inner coil covered by a first insulating sheath and an electrically conductive outer coil covered by a second insulating sheath, wherein said outer coil covered by said second insulating sheath is positioned coaxially over said inner coil covered by said first insulating sheath and said central portion has a length within a range of 390 mm to 590 mm, further wherein a connector is attached to and in electrical communication with said proximal end of said central portion; said first prong comprises said inner coil covered by said first insulating sheath and does not comprise said outer coil covered by said second insulating sheath, wherein said first prong has a length within a range of 50 mm to 120 mm, further wherein a first anchoring element and a first electrode are attached to said first prong and are positioned proximate said distal end of said first prong, said first anchoring element configured to permit the ingrowth of biological tissues; said second prong comprises said outer coil covered by said second insulating sheath and does not comprises said inner coil covered by said first insulating sheath, wherein said second prong has a length within a range of 50 mm to 120 mm, further wherein a second anchoring element and a second electrode are attached to said second prong and positioned proximate said distal end of said second prong, said second anchoring element configured to permit the ingrowth of biological tissues; and, a length of suturing material having a first end and a second end, wherein said first end of said length of suturing material is attached to said distal end of said first prong and said second end of said length of suturing material is attached to said distal end of said second prong, joining said first and second prongs, said length of suturing material forming a loop.

The length of suturing material may be in a range of 10 to 150 mm.

Optionally, wherein a distal end of said outer coil is positioned at said distal end of said central portion, said lead further comprises an additional electrically conductive coil having a proximal end and a distal end and comprising said second prong, wherein said proximal end of said additional coil is attached to said distal end of said outer coil and said distal end of said additional coil is attached to said second end of said length of suturing material, further wherein said second anchoring element and said second electrode are attached to and positioned proximate said distal end of said additional coil and said second insulating sheath extends over said additional coil.

The present specification also discloses a method of implanting an electrical stimulation lead having a connector, a first branch with a first electrode and first anchoring element and a second branch with a second electrode and second anchoring element, into a patient, said method comprising the steps of: inserting a distal end of an endoscope into a natural orifice of said patient; inserting a lead delivery catheter into a working channel of said endoscope, said lead delivery catheter comprising: a catheter body having a proximal end, a distal end, and a lumen within; an inflatable balloon attached to said distal end of said catheter body; and, a grasping mechanism attached to said distal end of said catheter body for grasping said lead; creating an incision in the internal wall of a body cavity entered via said orifice; advancing said distal end of said catheter through said incision into a target anatomy area, wherein said target anatomy area comprises the outer walls of the esophagus and stomach and surrounding tissues proximate the gastroesophageal junction (GEJ); inserting a laparoscope having a proximal end, a distal end, and a lumen within into an abdomen of said patient such that said distal end is positioned proximate said target anatomy area; placing said lead within said lumen of said laparoscope through said proximal end of said laparoscope; pulling on said loop of said lead via said grasping mechanism on said catheter to draw said lead into said target anatomy area; positioning the first branch and the second branch of said lead such that said first and second electrodes are positioned proximate the target anatomy; positioning said first anchoring element and said second anchoring element proximate surrounding tissues to permit growth of said surrounding tissues into said anchoring elements to secure said branches; and, attaching said connector of said lead to an electrical pulse generator.

The aforementioned and other embodiments of the present invention shall be described in greater depth in the drawings and detailed description provided below.

### BRIEF DESCRIPTION OF THE DRAWINGS

These and other features and advantages of the present invention will be further appreciated, as they become better understood by reference to the detailed description when considered in connection with the accompanying drawings:

FIG. 1A is a side view illustration of one embodiment of an implantable electrical stimulation lead of the present specification;

FIG. 1B is an oblique side view illustration of the embodiment of the implantable electrical stimulation lead of FIG. 1A;

FIG. 2 is a close-up view illustration of the first and second monopolar branches of the embodiment of the implantable electrical stimulation lead of FIG. 1A;

FIG. 3 is a close-up view illustration of the anchors and insulated proximal portions of the electrodes of the monopolar branches of the embodiment of the implantable electrical stimulation lead of FIG. 1A;

FIG. 4 is a close-up view illustration of the lengths of suture material attached to the distal ends of the monopolar branches of the embodiment of the implantable electrical stimulation lead of FIG. 1A;

FIG. 5A is a close-up view illustration of one embodiment of a needle used to suture in place the anchors of the implantable electrical stimulation leads of the present specification;

FIG. 5B is an oblique side view illustration of another embodiment of a needle used to suture in place the anchors of the implantable electrical stimulation leads of the present specification;

FIG. 5C is a side view illustration of the needle of FIG. 5B;

FIG. 5D is a cross-sectional illustration of the proximal end of the needle of FIG. 5A and FIG. 5B, in accordance with some embodiments of the present specification;

FIG. 6 is a side view illustration of another embodiment of an implantable electrical stimulation lead, depicting a length of suture material joining the distal ends of the two monopolar branches;

FIG. 7A is a side view illustration of one embodiment of an in-line bipolar implantable electrical stimulation lead;

FIG. 7B is an oblique side view illustration of one embodiment of an in-line bipolar implantable electrical stimulation lead;

FIG. 8 is an exploded side view illustration of one embodiment of an in-line bipolar implantable electrical stimulation lead;

FIG. 9 is a side view illustration of an embodiment of an in-line bipolar implantable electrical stimulation lead having a needle attached at its distal end;

FIG. 10 is a side view illustration of one embodiment of a lead delivery catheter used to implant a needleless electrical stimulation lead using the natural orifice transluminal endoscopic surgery (NOTES) technique;

FIG. 11 is a flowchart illustrating one embodiment of the steps involved in implanting a needleless electrical stimulation lead using an endoscope; and,

FIG. 12 is a flowchart illustrating one embodiment of the steps involved in endoscopically implanting an in-line bipolar electrical stimulation lead; and

FIG. 13 is a flowchart illustrating one embodiment of the steps involved in a method of implanting an electrical stimulation lead having a connector and a plurality of in-line electrodes into a patient.

### DETAILED DESCRIPTION

The present specification discloses an implantable electrical stimulation lead that is dimensioned specifically for use in confined anatomy, particularly the area proximate the gastroesophageal junction (GEJ). The lead is designed to be implanted laparoscopically and includes needles for suturing anchoring elements to the neighboring anatomy. The present specification also discloses another, needleless implantable electrical stimulation lead that is designed to be implanted through the working channel of an endoscope and includes anchoring elements that eliminate the need for suturing the lead to surrounding tissues. The present specification also discloses an in-line bipolar implantable electrical stimulation lead. In one embodiment, the in-line bipolar implantable electrical stimulation lead includes a suture loop at its distal end and is designed to be implanted endoscopically. In another embodiment, the in-line bipolar implantable electrical stimulation lead includes a needle at its distal end, rather than a loop, and is designed to be implanted laparoscopically. The present specification also discloses a lead delivery catheter used for implanting the needleless electrical stimulation lead through the working channel of an endoscope.

The present invention is directed toward multiple embodiments. The following disclosure is provided in order to enable a person having ordinary skill in the art to practice the invention. Language used in this specification should not be interpreted as a general disavowal of any one specific embodiment or used to limit the claims beyond the meaning of the terms used therein. The general principles defined herein may be applied to other embodiments and applications without departing from the spirit and scope of the invention. Also, the terminology and phraseology used is for the purpose of describing exemplary embodiments and should not be considered limiting. Thus, the present invention is to be accorded the widest scope encompassing numerous alternatives, modifications and equivalents consistent with the principles and features disclosed. For purpose of clarity, details relating to technical material that is known in the technical fields related to the invention have not been described in detail so as not to unnecessarily obscure the present invention.

In one embodiment, an implantable electrical stimulation lead is a bipolar lead and comprises an elongate lead body having a proximal end and a distal end. The lead body is comprised of an electrically conductive material with an overlaying insulating sheath. Attached to the proximal end is a coupling means for connecting the lead to a pulse generator such that the two are in electrical communication. In one embodiment, the coupling means is an international standard (IS-1) connector system. In various embodiments, the entire lead body is insulated and the coupling means or connector system is not insulated. The distal end of the lead body includes a bifurcation sleeve. In one embodiment, the electrically conductive material of the lead body includes an inner coil and an outer coil, electrically insulated from each other, which split into separate branches within the bifurcation sleeve.

The inner coil and outer coil continue distally beyond the bifurcation sleeve as first and second monopolar branches. In one embodiment, the first and second monopolar branches comprise first and second elongate branch bodies respectively, each having a proximal end and a distal end. In one embodiment, the first branch body of the first monopolar branch comprises the continuation of the inner coil of the lead body and the second branch body of the second monopolar branch comprises a partial continuation of the outer coil of lead body attached to an additional coil. The additional coil is an elongate coil having a proximal end and a distal end with its proximal end attached to the distal end of the outer coil. In another embodiment, the first branch body of the first monopolar branch comprises the continuation of the inner coil of the lead body and the second branch body of the second monopolar branch comprises the continuation of the outer coil of lead body. The proximal ends of the first and second branch bodies join together within the bifurcation sleeve as described above. The distal ends of the first and second branch bodies each have a length of suturing material attached to them. In one embodiment, the suture is a monofilament using nylon as the material. In another embodiment, the suture is multi-filament. In one embodiment, the suture is resorbable. In another embodiment, the suture is non-resorbable. Attached to the distal end of each length of suturing material is a needle. In one embodiment, the needle is a curved needle. In one embodiment, the needle is a straight needle. Both the first and second branch bodies additionally include at least one anchor and at least one electrode. Each electrode is in electrical communication with either the inner or outer coil of its respective branch body. In one embodiment, the anchor has a butterfly shape with two holes, one on each side, for passing the needle and suture material during anchoring. Each electrode is positioned just distal to each anchor. In one embodiment, the first monopolar branch has a length that is longer than that of the second monopolar branch. In another embodiment, the first and second monopolar branches have the same length.

In one embodiment, a portion of each electrode is insulated by a length of tubing. In one embodiment, the tubing extends distally from the distal end of the anchoring element. In one embodiment, the tubing and anchoring element are composed of silicone.

In various embodiments, the entirety of each branch is insulated with the exception of each electrode or a portion of each electrode.

The lead is designed to be implanted using a standard laparoscopic technique common in the prior art.

In another embodiment, an implantable electrical stimulation lead is intended for implantation via the working channel of an endoscope and includes anchoring elements rather than a needle and sutures for anchoring. In this embodiment, the implantable electrical stimulation lead is a bipolar lead and also comprises an elongate lead body having a proximal end and a distal end. The lead body is comprised of an electrically conductive material with an overlaying insulating sheath. Attached to the proximal end is a coupling means for connecting the lead to a pulse generator such that the two are in electrical communication. In one embodiment, the coupling means is an IS-1 connector system. The distal end of the lead body includes a bifurcation sleeve. In one embodiment, the electrically conductive material of the lead body includes an inner coil and an outer coil, electrically insulated from each other, which split into separate branches within the bifurcation sleeve.

The inner coil and outer coil continue distally beyond the bifurcation sleeve as first and second monopolar branches. In one embodiment, the first and second monopolar branches comprise first and second elongate branch bodies respectively, each having a proximal end and a distal end. In one embodiment, the first branch body of the first monopolar branch comprises the continuation of the inner coil of the lead body and the second branch body of the second monopolar branch comprises a partial continuation of the outer coil of lead body attached to an additional coil. The additional coil is an elongate coil having a proximal end and a distal end with its proximal end attached to the distal end of the outer coil. In another embodiment, the first branch body of the first monopolar branch comprises the continuation of the inner coil of the lead body and the second branch body of the second monopolar branch comprises the continuation of the outer coil of lead body. The proximal ends of the first and second branch bodies join together within the bifurcation sleeve as described above. The distal ends of the first and second branch bodies are connected by a suture loop. The suture loop is designed to be grasped with endoscopic graspers and pulled through the working channel of the endoscope. In one embodiment, the material of the suture loop is silk. Both the first and second branch bodies additionally include at least one anchoring element and at least one electrode. Each electrode is in electrical communication with either the inner or outer coil of its respective branch body. The anchoring elements allow for fibrosis around them in the created endoscopic tunnel so that the electrodes remain in position. This eliminates the need for suturing the lead branches in place. In various embodiments, the anchoring element is a silicone sleeve having grooves, spikes, or holes to allow for the ingrowth of fibrous tissue and anchoring. In another embodiment, the anchoring element is comprised of a porous material that allows fibrous ingrowth and anchoring. In one embodiment, the porous material is a Dacron mesh. In another embodiment, the anchoring material is made of an electrically conductive material, such as platinum-iridium alloy, and is electrically connected to the electrode to increase the area of stimulation. In another embodiment, the electrodes are the anchors, with special shapes, such as barbs, to facilitate anchoring and tissue in-growth. Each electrode is positioned just distal to each anchor. In one embodiment, the first monopolar branch has a length that is longer than that of the second monopolar branch such that the electrodes are staggered in an in-line position. In another embodiment, the first and second monopolar branches have the same length.

The present specification also discloses a lead delivery catheter for use during the implantation of the needleless electrical stimulation lead through the working channel of an endoscope. In one embodiment, the catheter is used with the natural orifice transluminal endoscopic surgery (NOTES) technique to implant one or more leads proximate the lower esophageal sphincter (LES) using an endoscopic approach or a laparoscopic approach. In one embodiment, the catheter includes a catheter body having a proximal end, a distal end, and a lumen within. The catheter includes an inflatable balloon, a grasping mechanism, and a light source at its distal end. Optionally, in one embodiment, the catheter includes a camera at its distal end. Optionally, in one embodiment, the catheter includes a bipolar electrode at its distal end for electrocautery.

The leads disclosed in the various embodiments of the present specification can be implanted into a patient using the methods described in U.S. patent application Ser. No. 13/602,184, entitled “Endoscopic Lead Implantation Method”, filed on Sep. 2, 2012, and assigned to the applicant of the present invention, which is herein incorporated by reference in its entirety.

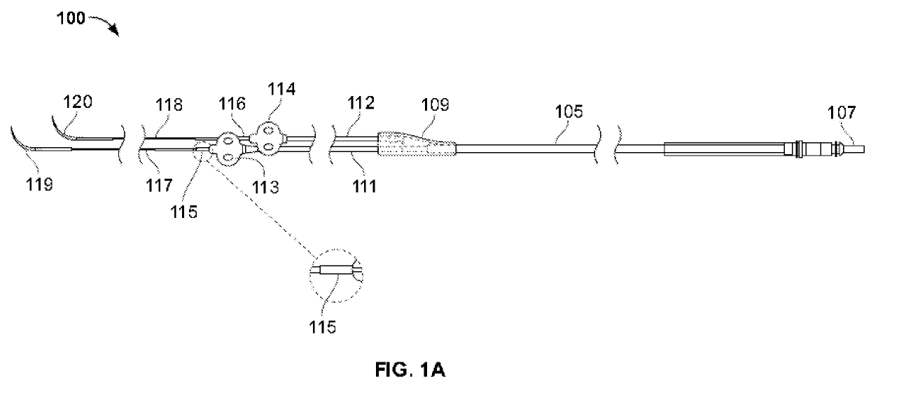

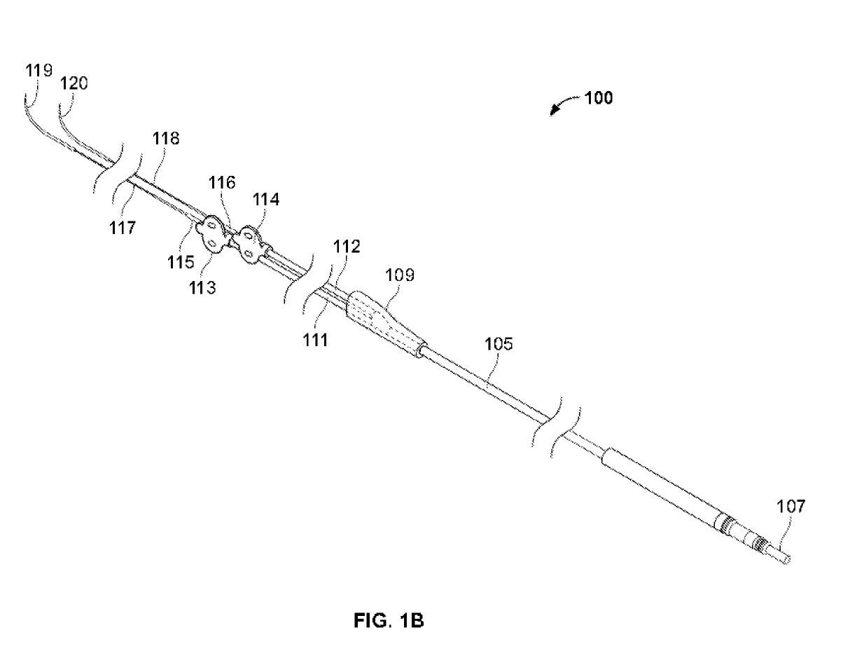

FIGS. 1A and 1B are side and oblique side view illustrations respectively, of one embodiment of an implantable electrical stimulation lead 100 of the present specification. The lead 100 is a bipolar lead and includes an elongate lead body 105 having a proximal end and a distal end. The lead body 105 is comprised of an electrically conductive inner coil and an electrically conductive outer coil. The inner coil and outer coil are each covered by an insulating sheath. An IS-1 connector system 107, having proximal and distal ends, is attached to the proximal end of the lead body 105 and a bifurcation sleeve 109, having proximal and distal ends, is coupled to the distal end of the lead body 105. In various embodiments, the length of the lead body 105, from the proximal end of the IS-1 connector pin 107 to the distal end of the bifurcation sleeve 109, is in a range of 390 mm to 590 mm. In one embodiment, the length of the lead body 105, from the proximal end of the IS-1 connector pin 107 to the distal end of the bifurcation sleeve 109, is 433 mm. This length is greater than that encountered in the prior art, which often measures approximately 350 mm. The greater length allows for greater variation in implantation site. A physician can implant the lead from a more cosmetically pleasing position, for example, a sub-bikini line implantation site or a transumbilical implantation site. The resulting stimulator implant scar would not be visible on the patient's abdomen. In addition, the greater length allows for appropriate routing of the lead to prevent entanglement in the small bowel or a gravid uterus in a female with child bearing potential.

The inner and outer coils of the lead body 105 separate within the bifurcation sleeve 109 and continue distally as monopolar branches. Referring to FIGS. 1A and 1B, the inner coil continues distally from the distal end of the bifurcation sleeve 109 as a first monopolar branch 111, having proximal and distal ends, and the outer coil continues distally from the distal end of the bifurcation sleeve 109 and attaches to an additional coil having proximal and distal ends, which continues as a second monopolar branch 112 having proximal and distal ends. In another embodiment, the outer coil continues distally from the distal end of the bifurcation sleeve 109 as the second monopolar branch 112 having proximal and distal ends. The first monopolar branch 111 comprises the inner coil with a covering insulating sheath and includes an anchor 113, having a proximal end and a distal end, and an insulated electrode 115, having a proximal end and a distal end, at a point proximate its distal end. The electrode 115 is positioned just distal to the anchor 113. Attached to the distal end of the first monopolar branch 111 is a length of suture material 117, itself having a proximal end and a distal end. In one embodiment, the suture material is composed of nylon. Attached to the distal end of the suture material is a suture needle 119. The second monopolar branch 112 comprises a portion of the outer coil and an attached additional coil with a covering insulating sheath and includes an anchor 114, having a proximal end and a distal end, and an insulated electrode 116, having a proximal end and a distal end, at a point proximate its distal end. The electrode 116 is positioned just distal to the anchor 114. Attached to the distal end of the second monopolar branch 112 is a length of suture material 118, itself having a proximal end and a distal end. In one embodiment, the suture material is composed of nylon. Attached to the distal end of the suture material is a suture needle 120.

In another embodiment, each branch includes an additional suture with needle and the anchor, in a butterfly shape, is positioned just distal to the bifurcation sleeve. The additional suture and position of the anchor will help maintain the anchor flat on the esophagus after implantation. This will prevent the anchor from pivoting and avoid extra pressure on the esophageal wall.

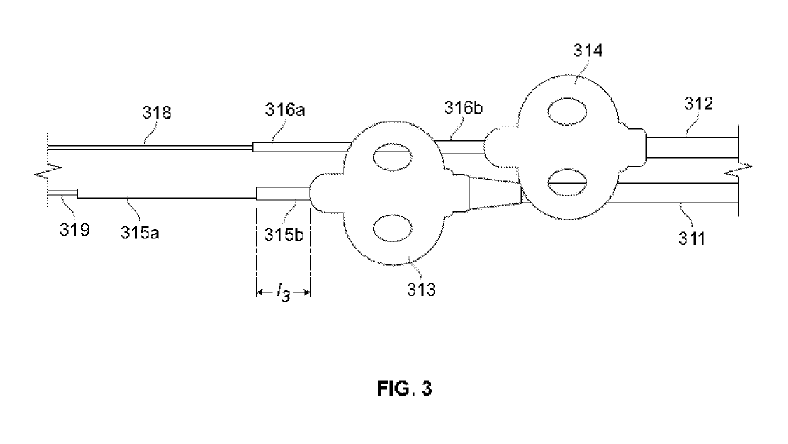

FIG. 1A also includes a close-up view illustration of the insulated electrode 115 of the first monopolar branch 111. In one embodiment, the electrode 115 includes a covering length of insulating material which will be discussed further with reference to FIG. 3 below. In another embodiment, the electrode is not covered by any insulating material.

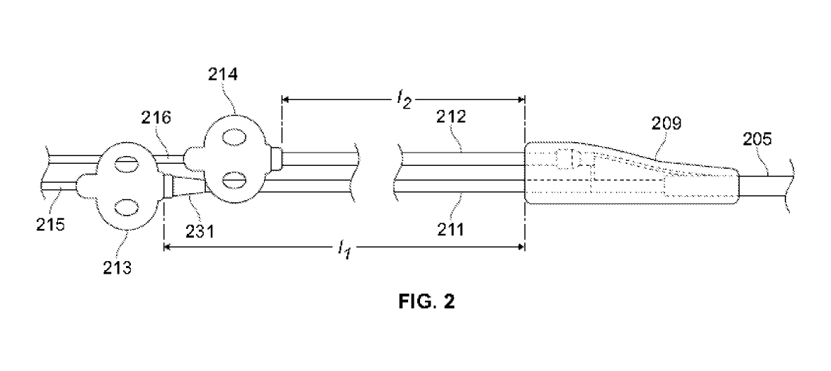

FIG. 2 is a close-up view illustration of the first 211 and second 212 monopolar branches of the embodiment of the implantable electrical stimulation lead of FIG. 1A. The monopolar branches 211, 212 are depicted emanating distally from the distal end of the bifurcation sleeve 209. Also depicted is the distal end of the lead body 205 coupled to the bifurcation sleeve 209. The first monopolar branch 211 includes an anchor 213 and an insulated electrode 215 at a point proximate its distal end and the second monopolar branch 212 includes an anchor 214 and an insulated electrode 216 at a point proximate its distal end. In various embodiments, the length l₁ of the first monopolar branch 211, from the tip of its proximal end where it exits the distal end of the bifurcation sleeve 209 to the tip of its distal end where it meets the proximal end of the anchor 213, is in a range of 20 mm to 150 mm and more preferably, 50 mm to 120 mm. In one embodiment, the length l₁ of the first monopolar branch 211, from the tip of its proximal end where it exits the distal end of the bifurcation sleeve 209 to the tip of its distal end where it meets the proximal end of the anchor 213, is 70 mm. This is shorter than the length encountered in the prior art, which is approximately 90 mm. In various embodiments, the length l₂ of the second monopolar branch 212, from the tip of its proximal end where it exits the distal end of the bifurcation sleeve 209 to the tip of its distal end where it meets the proximal end of the anchor 214, is in a range of 20 mm to 150 mm and more preferably, 50 mm to 120 mm. In one embodiment, the length l₂ of the second monopolar branch 212, from the tip of its proximal end where it exits the distal end of the bifurcation sleeve 209 to the tip of its distal end where it meets the proximal end of the anchor 214, is 60 mm. This is shorter than the length encountered in the prior art, which is approximately 90 mm.

The longer length of the monopolar branches in the prior art facilitates their implantation across the gastric greater curvature, with one electrode on each wall. The shorter lengths of the monopolar branches of the lead of the current embodiment facilitate placement about the GEJ, where the anatomy in more confined. In one embodiment, the first monopolar branch 211 further includes a visual indicator 231 at its distal end, just proximal to the anchor 213. The visual indicator 231 indicates to the physician that this lead contains the inner coil of the lead body. In one embodiment, the visual indicator 231 is a black marking on the insulation of the first monopolar branch 211. Having monopolar branches of different lengths allows the physician to implant the electrodes in-line with each other.

FIG. 3 is a close-up view illustration of the anchors 313, 314 and insulated proximal portions of the electrodes 315b, 316b of the monopolar branches 311, 312 of the embodiment of the implantable electrical stimulation lead of FIG. 1A. In one embodiment, the electrode of the first monopolar branch 311 comprises an exposed portion 315a and an insulated, unexposed portion 315b that is covered by a length of insulating tubing. In various embodiments, the length l₃ of the insulating tubing covering the insulated portion of the electrode 315b is in a range of 1 mm to 10 mm and more preferably, 1 mm to 5 mm. In one embodiment, the length l₃ of the insulating tubing covering the insulated portion of the electrode 315b is 3 mm. In one embodiment, the insulating tubing is attached to the distal end of the anchor 313. Depicted attached to the distal end of the exposed portion of the electrode 315a is the proximal end of a length of suture material 319. In another embodiment, the electrode of the first monopolar branch does not include any insulating tubing and is exposed along its entire length (not shown).

In one embodiment, the electrode of the second monopolar branch 312 comprises an exposed portion 316a and an insulated, unexposed portion 316b that is covered by a length of insulating tubing. In various embodiments, the length of the insulating tubing covering the insulated portion of the electrode 316b of the second monopolar branch 312 is the same as the length of the insulating tubing covering the insulated portion of the electrode 315b of the lead of the first monopolar branch 311, that is, in a range of 1 mm to 10 mm and more preferably, 1 mm to 5 mm. In one embodiment, the length of the insulating tubing covering the insulated portion of the electrode 316b of the second monopolar branch 312 is the same as the length of the insulating tubing covering the insulated portion of the electrode 315b of the lead of the first monopolar branch 311, that is, 3 mm. In one embodiment, the insulating tubing covering the insulated portion of the electrode 316b is attached to the distal end of the anchor 314. Depicted attached to the distal end of the exposed portion of the electrode 316a is the proximal end of a length of suture material 318. In another embodiment, the electrode of the second monopolar branch does not include any insulating tubing and is exposed along its entire length (not shown).

The insulating tubing covering the insulated, unexposed portions of the electrodes 315b, 316b serve to prevent the exposure of the proximal 2 to 3 mm of each electrode that often occurs during anchoring as the electrodes are pulled backward slightly over time.

In one embodiment, the insulating tubing covering the insulated portions of the electrodes 315b, 316b is composed of silicone. In various embodiments, the wall thickness of the insulating tubing is in a range of 0.130 mm to 0.200 mm and more preferably, 0.160 mm to 0.170 mm. In one embodiment, the wall thickness of the insulating tubing is 0.165 mm (0.0065 in). In one embodiment, the anchors 313, 314 are composed of silicone. In one embodiment, the electrodes are composed of platinum-iridium (Pt—Ir). In various embodiments, the exposed portion of the electrodes 315a, 316a, after anchoring, is in a range of 1 mm to 20 mm and more preferably, 1 mm to 10 mm. In one embodiment, the exposed portion of the electrodes 315a, 316a, after anchoring, is 5 mm. This length is shorter than the average of approximately 10 mm encountered in the prior art. The shorter electrodes have a higher charge density which has been shown to contribute to better results.

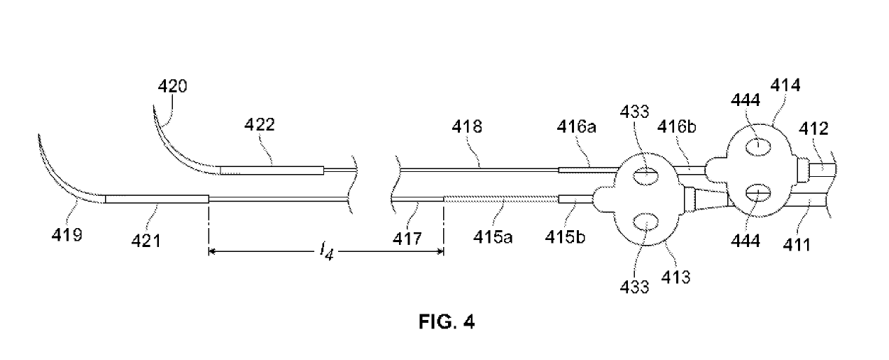

FIG. 4 is a close-up view illustration of the lengths of suture material 417, 418 attached to the distal ends of the monopolar branches 411, 412 of the embodiment of the implantable electrical stimulation lead of FIG. 1A. Also depicted are the anchors 413, 414, exposed electrode portions 415a, 415b, and insulating tubing covering the insulated portions of the electrodes 415b, 416b of the first 411 and second 412 monopolar branches. Attached to the distal end of the first monopolar branch 411 and extending distally from the exposed portion of electrode 415a is a first length of suture material 417. The length of suture material 417 includes a proximal end and a distal end. A suture needle 419 is attached to the distal end of the suture material 417 via a coupling means 421. In various embodiments, the length l₄ of the suture material 417 is in a range of 40 mm to 80 mm and more preferably, 55 mm to 65 mm. In one embodiment, the length l₄ of the suture material 417 is 60 mm.

Attached to the distal end of the second monopolar branch 412 and extending distally from the exposed portion of electrode 416a is a second length of suture material 418. The length of suture material 418 includes a proximal end and a distal end. A suture needle 420 is attached to the distal end of the suture material 418 via a coupling means 422. In various embodiments, the length of the suture material 418 attached to the distal end of the second monopolar branch 412 is the same as the length of the suture material 417 attached to the distal end of the first monopolar branch 411, that is, in a range of 40 mm to 80 mm and more preferably, 55 mm to 65 mm. In one embodiment, the length of the suture material 418 attached to the distal end of the second monopolar branch 412 is the same as the length of the suture material 417 attached to the distal end of the first monopolar branch 411, that is, 60 mm.

The average length of the suture material encountered in leads in the prior art is approximately 112 mm. For applications at the GEJ, such a length requires the physician to perform additional, unnecessary pulling maneuvers in order to properly position the anchors. The area to maneuver proximate the GEJ is limited by the proximity of the GEJ to the diaphragm. Therefore, a lead with shorter lengths of suture material is advantageous for such an application.

In one embodiment, the suture material is composed of nylon. In another embodiment, the suture material is barbed, such as V-Loc™ by Covidien, to improve anchoring of the electrodes. During anchoring, a physician sutures the branches into position by threading the needles 419, 420 through holes 433, 444 in the anchors 413, 414 and into the surrounding tissue. In one embodiment, the anchors 413, 414 have a butterfly shape with two holes 433, 444 positioned on either side of each monopolar branch 411, 412.

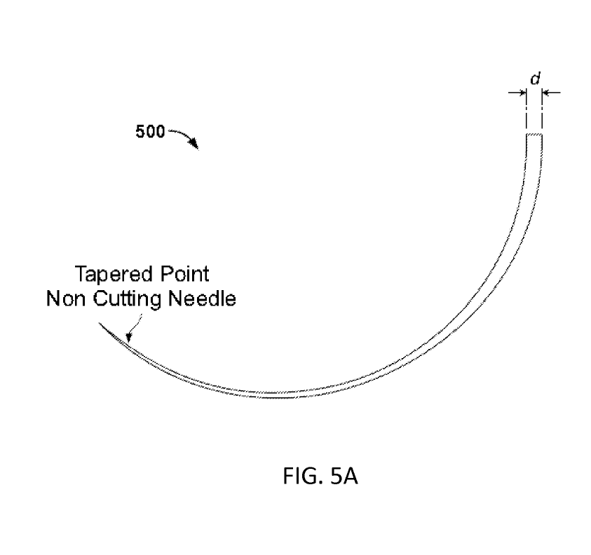

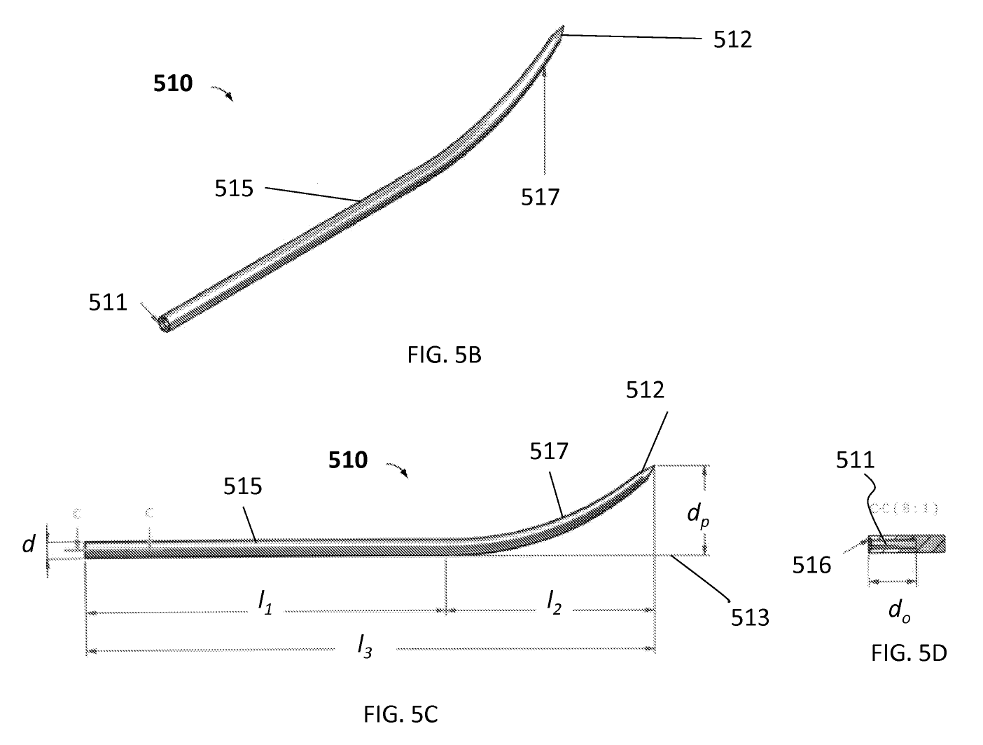

FIG. 5A is a close-up view illustration of one embodiment of a needle 500 used to suture in place the anchors of the implantable electrical stimulation leads of the present specification. A needle 500 is attached to the distal end of each length of suture material emanating from the distal end of each monopolar branch. In one embodiment, each needle 500 is attached to the distal end of the suture material via a coupling means. In one embodiment, each needle 500 is a ⅜ of a circle curve needle and has a length within a range of 13 mm to 28 mm and more preferably, 18 to 23 mm. In another embodiment, each needle 500 is a ¼ of a circle curve needle and has a length within a range of 13 mm to 28 mm and more preferably, 18 to 23 mm. The needle 500 has a tapered point and is a non-cutting needle. In various embodiments, the needle has a diameter d at its base in a range of 0.58 mm to 0.88 mm and more preferably, 0.68 mm to 0.78 mm, being at least as large as the diameter of the insulated or non-insulated electrode. In one embodiment, the needle has a diameter d at its base of 0.73 mm (0.029 in), which is 0.56 mm (0.022 in) larger than the insulating tubing of the electrode.

FIGS. 5B and 5C are oblique side view and side view illustrations respectively, of another embodiment of a needle 510 used to suture in place the anchors of the implantable electrical stimulation leads of the present specification. A needle 510 is attached to the distal end of each length of suture material emanating from the distal end of each monopolar branch. In one embodiment, referring to FIGS. 5B and 5C, each needle 510 includes a straight proximal portion 515 and a curved distal portion 517. In various embodiments, the curved distal portion 517 has a radius within a range of 6 mm to 13 mm, more preferably 8 mm to 11 mm, and even more preferably a radius of 9.67 mm. In various embodiments, a length l₁ of the straight proximal portion 515 is within a range of 8 mm to 16 mm and more preferably, 11 mm to 13 mm, and a length l₂ of the curved distal portion 517 is within a range of 4 mm to 10 mm and more preferably, 6 mm to 8 mm, resulting in an overall length l₃ of the needle 510 within a range of 12 mm to 26 mm and more preferably, 17 mm to 21 mm. In one embodiment, a length l₁ of the straight proximal portion 515 is 12 mm and a length l₂ of the curved distal portion 517 is 7 mm, resulting in an overall length l₃ of the needle 510 of 19 mm. The needle 510 includes a tapered point 512 at its distal end and is a non-cutting needle. In various embodiments, the curve of the needle 510 is such that the tapered point 512 is positioned a distance dₚ within a range of 1 mm to 5 mm and more preferably, 2 mm to 4 mm, from an axis 513 of the proximal straight portion 515. In one embodiment, the curve of the needle 510 is such that the tapered point 512 is positioned a distance dₚ of 3 mm from an axis 513 of the proximal straight portion 515. In various embodiments, the needle has a diameter d at its base in a range of 0.41 mm to 0.71 mm and more preferably, 0.51 mm to 0.61 mm. In one embodiment, the needle has a diameter d at its base of 0.56 mm.

In various embodiments, the needle 510 is attached to the distal end of a length of suture material via a coupling means. In one embodiment, the coupling means comprises a hole or opening 511 in the proximal end of the needle 510. FIG. 5D is a cross-sectional illustration of the proximal end of the needle of FIGS. 5A and 5B, in accordance with some embodiments of the present specification. FIG. 5D illustrates a section C-C at the proximal end of the needle 510 as seen in FIG. 5C. Referring to FIGS. 5B-5D simultaneously, the opening 511 extends into the proximal end of the needle 510 at least a distance dₒ of 1.60 mm. In one embodiment, the needle 510 includes a beveled surface 516 at an angle of 45° and having a length of approximately 0.1 mm at the opening 511. In various embodiments, the opening 511 is configured to fixedly receive a length of suture. In some embodiments, the proximal end of the needle is crimped after the suture has been inserted into the opening 511 to secure the suture to the needle. In one embodiment, the opening is configured to fixedly receive a length of mononylon suture United States Pharmacopeia (USP) 3/0. In one embodiment, the needle 510 is composed of stainless steel 302 with a general tolerance of +/−0.1 mm.

During anchoring, the electrode tract should be straight. Traditional ½ curve sky shaped or ski needles encountered in the prior art start with a tight bend and hence require a circular maneuver. With such a needle, when a straight bite is attempted, the tissue is often heavily injured, similar to what occurs with a biopsy. The needle of the present embodiment, having a shorter curve, can be more easily straightened when maneuvering near the GEJ when compared to the needles of the prior art. In addition, suturing needles and leads encountered in the prior art often include a suture sleeve. Such sleeves tend to attract fibrosis. The lead of the present specification does not include a sleeve so as to minimize fibrosis.

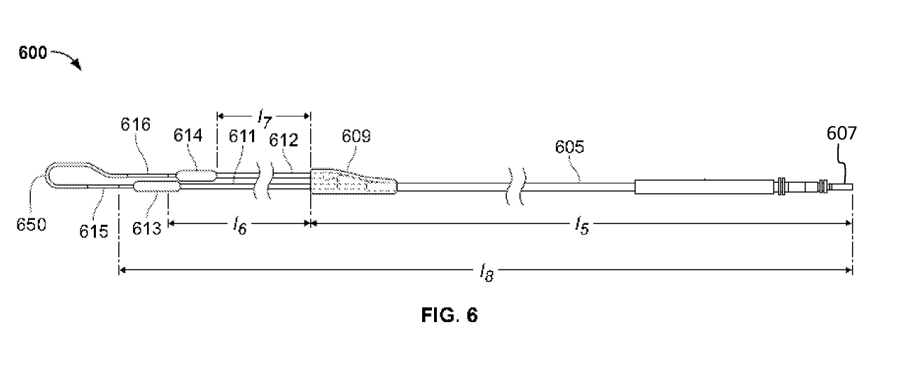

FIG. 6 is a side view illustration of another embodiment of an implantable electrical stimulation lead 600, depicting a length of suture material 650 joining the distal ends of the two monopolar branches 611, 612. The lead 600 is a bipolar lead and includes an elongate lead body 605 having a proximal end and a distal end. The lead body 605 is comprised of an electrically conductive inner coil and an electrically conductive outer coil. The outer coil is covered by an insulating sheath. An IS-1 connector system 607, having proximal and distal ends, is attached to the proximal end of the lead body 605 and a bifurcation sleeve 609, having proximal and distal ends, is coupled to the distal end of the lead body 605. In various embodiments, the length l₅ of the lead body 605, from the proximal end of the IS-1 connector system 607 to the distal end of the bifurcation sleeve 609, is in a range of 390 mm to 590 mm. In one embodiment, the length l₅ of the lead body 605, from the proximal end of the IS-1 connector system 607 to the distal end of the bifurcation sleeve 609, is 433 mm.

The inner and outer coils of the lead body 605 separate within the bifurcation sleeve 609 and continue distally as monopolar branches. The inner coil continues distally from the distal end of the bifurcation sleeve 609 as a first monopolar branch 611, having proximal and distal ends, and a portion of the outer coil continues distally from the distal end of the bifurcation sleeve 609 and attaches to an additional coil, having proximal and distal ends, which continues as a second monopolar branch 612 having proximal and distal ends. In another embodiment, the outer coil continues distally from the distal end of the bifurcation sleeve 609 as the second monopolar branch 612 having proximal and distal ends. The first monopolar branch 611 comprises the inner coil with a covering insulating sheath and includes an anchor 613, having a proximal end and a distal end, and an electrode 615, having a proximal end and a distal end, at a point proximate its distal end. The electrode 615 is positioned just distal to the anchor 613. The second monopolar branch 612 comprises a portion of the outer coil and an attached additional coil with a covering insulating sheath and includes an anchor 614, having a proximal end and a distal end, and an electrode 616, having a proximal end and a distal end, at a point proximate its distal end. The electrode 616 is positioned just distal to the anchor 614. In various embodiments, the length l₆ of the first monopolar branch 611, from its proximal end where it exits the distal end of the bifurcation sleeve 609 to its distal end where it meets the proximal end of the anchor 613, is in a range of 20 mm to 150 mm and more preferably, 50 mm to 120 mm. In one embodiment, the length l₆ of the first monopolar branch 611, from its proximal end where it exits the distal end of the bifurcation sleeve 609 to its distal end where it meets the proximal end of the anchor 613, is 70 mm. In various embodiments, the length l₇ of the second monopolar branch 612, from its proximal end where it exits the distal end of the bifurcation sleeve 609 to its distal end where it meets the proximal end of the anchor 614, is in a range of 20 mm to 150 mm and more preferably, 50 mm to 120 mm. In one embodiment, the length l₇ of the second monopolar branch 612, from its proximal end where it exits the distal end of the bifurcation sleeve 609 to its distal end where it meets the proximal end of the anchor 614, is 60 mm.

In various embodiments, the length of the electrodes 615, 616 is in a range of 1 mm to 20 mm and more preferably, 1 mm to 10 mm. In one embodiment, the length of the electrodes 615, 616 is 5 mm. The different lengths of the first and second monopolar branches allow the electrodes to be positioned in a staggered, in-line configuration. In various embodiments, after anchoring, the electrodes are positioned in a range of 1 to 40 mm and more preferably, 1 to 20 mm, apart from one another. In one embodiment, after anchoring, the electrodes are positioned 10 mm apart from one another.

A length of suture material 650, having a first end and a second end, joins the two monopolar branches 611, 612. The first end of the length of suture material 650 is attached to the distal end of the first monopolar branch 611, just distal to the electrode 615, and the second end of the length of suture material 650 is attached to the distal end of the second monopolar branch 612, just distal to the electrode 616. The suture material 650 acts as a loop to direct the lead 600 during implantation. In various embodiments, the suture material has a length of 10 to 150 mm. In one embodiment, the suture material has a length of 60 mm. In one embodiment, the suture material 650 is composed of nylon. In various embodiments, the total length of the lead 600 from the proximal end of the IS-1 connector system 607 to the proximal end of the electrode 615 of the first monopolar branch 611 is in a range of 500 mm to 540 mm. In one embodiment, the total length of the lead 600 from the proximal end of the IS-1 connector system 607 to the proximal end of the electrode 615 of the first monopolar branch 611 is 520 mm.

The implantable electrical implantation lead 600 is designed to be implanted through the working channel of an endoscope. A physician inserts an endoscope into a patient using natural orifice transluminal endoscopic surgery (NOTES). In NOTES, a physician passes an endoscope through a natural orifice in the patient's body, such as, the mouth, urethra, or anus, creates an incision in the wall of an internal organ, such as, the stomach, bladder, or colon, and then passes the endoscope through the incision and into the target area or lumen of the organ. The incision is always internal with a NOTES technique, therefore, no visible scar remains. For the present embodiment, once the distal end of the endoscope is positioned proximate the target anatomy, the physician uses endoscopic graspers to grasp the suture material 650 of the lead 600 and then pulls the lead 600 through the working channel of the endoscope. Alternatively, the lead could be passed through a working channel of a laparoscopic and pulled through the endoscopic tunnel proximate to the target tissue thus eliminating the need to dissect to expose the target tissue. The monopolar branches 611, 612 are then positioned proximate the target anatomy. The anchors 613, 614 are designed to allow for fibrosis around the implantation site in the endoscopic tunnel, thereby holding the electrodes 615, 616 in place and eliminating the need for needles and sutures. In various embodiments, the anchors 613, 614 comprise sleeves having grooves, spikes, or holes to allow for the ingrowth of fibrous tissue and resultant anchoring. In another embodiment, the anchors are narrow plastic strips having a plurality of openings for tissue ingrowth. In another embodiment, the anchors are porous silicone with a plurality of openings for tissue ingrowth and neovascularization. In another embodiment, the anchors are rosette-shaped and include a plurality of openings for tissue ingrowth. In various embodiments, the anchors are configured to be wide enough to perform as stoppers but are sufficiently fluffy (porous) to prevent erosion through the esophageal wall. In one embodiment, the anchors are comprised of silicone. In another embodiment, the anchors 613, 614 are composed of a porous material that promotes fibrosis and anchoring. In one embodiment, the anchors are comprised of a Dacron mesh.

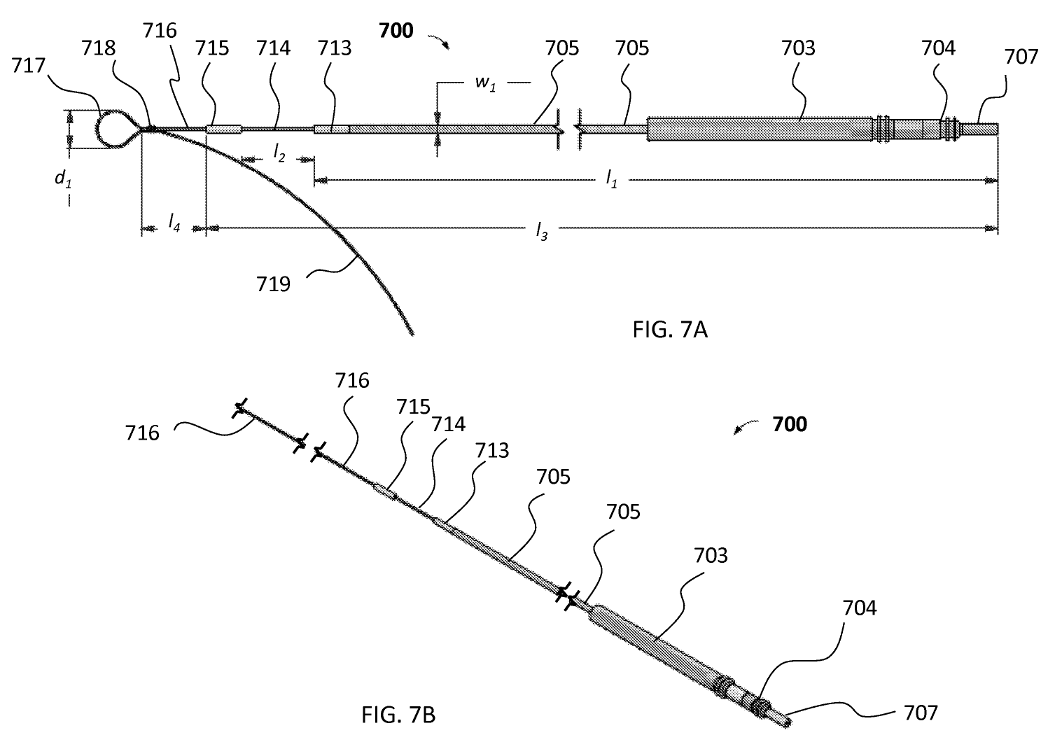

FIGS. 7A and 7B are side and oblique view illustrations respectively, of one embodiment of an in-line bipolar implantable electrical stimulation lead 700. A ‘unibody’ lead, wherein the electrodes are arranged in-line, provides several benefits over a lead having multiple branches. Firstly, since the physician is only manipulating one elongate lead body, a unibody lead is much easier to handle and deliver via an endoscopic approach. Secondly, there is only one anchoring step required during implantation, rather than multiple anchoring steps, one for each branch, with a multi-branch lead. Thirdly, there is only one possible point of migration and/or erosion with a unibody lead, rather than multiple migration/erosion points (again, one for each branch) encountered with a multi-branch lead.

Referring to FIGS. 7A and 7B simultaneously, lead 700 is an in-line bipolar lead and includes a flexible, elongate lead body 705 having a proximal end and a distal end. In one embodiment, the lead body 705 is comprised of a plurality of insulated electrically conductive coils. In another embodiment, the lead body 705 is comprised of a plurality of insulated electrically conductive cables. In another embodiment, the lead body 705 is comprised of one insulated electrically conductive coil. In various embodiments, the coils or cables are comprised of MP35N LT alloy. In various embodiments, the width w₁ of the lead body 705 is in a range of 0.20 mm to 2.00 mm and more preferably, 0.50 mm to 1.70 mm. In one embodiment, the width w₁ of the lead body 705 is 1.09 mm. A connector system 707, having proximal and distal ends, is attached to the proximal end of the lead body 705. In one embodiment, the connector system 707 is a conventional IS-1 BI connector system similar to that used with many cardiac pacemakers. After lead 700 delivery, the connector system 707 is connected to an implantable pulse generator (IPG) to make the lead operational. In various embodiments, the entire lead body 705 is insulated and the connector system 707 is not insulated.

A connector sleeve 703 covers a proximal portion of the lead body 705 and a distal portion of the connector system 707. In one embodiment, the sleeve 703 is comprised of silicone. In another embodiment, the sleeve 703 is comprised of polyurethane. The sleeve 703 facilitates handling of the lead 700 by a user. A connector retention ring 704 secures the sleeve 703 to the connector system 707 and lead body 705. In one embodiment, the retention ring 704 is comprised of silicone. A first electrode 713 is positioned at the distal end of the lead body 705. In various embodiments, the length l₁ from the tip of the proximal end of the connector system 707 to the tip of the distal end of the first electrode 713 is in a range of 450 to 550 mm. In one embodiment, the length l₁ from the tip of the proximal end of the connector system 707 to the tip of the distal end of the first electrode 713 is 505.9 mm. Extending distally from the first electrode 713 and in-line with the first electrode 713 and lead body 705 is a length of insulated electrical conductor 714. In some embodiments, the conductor 714 comprises a coiled wire. In other embodiments, the conductor 714 comprises a cable. In some embodiments, the conductor 714 is comprised of a plurality of individual conductors, or, in other words, a plurality of individual coiled wires or individual cables. Positioned at the distal end of the conductor 714 is a second electrode 715. In various embodiments, the electrodes 713, 715 each have a length in a range from 1 to 25 mm and more preferably, 1 to 15 mm. In one embodiment, the electrodes 713, 715 each have a length of 10 mm. In various embodiments, the electrodes 713, 715 each have a diameter in a range from 0.10 to 1.50 mm and more preferably, 0.25 to 1 mm. In one embodiment, the electrodes 713, 715 have a diameter of 0.46 mm. In one embodiment, the electrodes 713, 715 are comprised of platinum-iridium. In various embodiments, the electrodes 713, 715 are comprised of platinum-iridium with an iridium oxide coating or platinum with various coatings, including, but not limited to, iridium oxide and titanium nitride. In various embodiments, the two electrodes 713, 715 are configured to function with one being the cathode and the other being the anode. The physician can control which electrode functions as cathode and which functions as anode. In another embodiment, a housing of the IPG can be the anode or the cathode, thus allowing either electrode to be cathode or anode.

In some embodiments, the conductor 714 is an extension of the material comprising the lead body 705 wherein each electrode 713, 715 is positioned coaxially about said conductor 714 and in electrical communication with said conductor 714. In other words, the lead 700 includes at least one conductor 714 positioned between and extending through each of said plurality of electrodes 713, 715, thereby connecting each of said plurality of electrodes 713, 715. In some embodiments, the conductor 714 comprises an outer conductor positioned coaxially over an inner conductor. In one embodiment, the outer conductor extends from the lead body 705 and connects to the first electrode 713 while the inner conductor extends further and connects to the second electrode 715. In one embodiment, the outer conductor is composed of MP35N LT (stainless steel alloy) and comprises a 0.003″ diameter wire coiled into 0.33 mm (inner diameter) structure. In one embodiment, the inner conductor comprises a DFT coated cable (7×7 conductor structure) and has an outer diameter of 0.33 mm. In various embodiments, each electrode 713, 715 comprises a 0.1 mm platinum-iridium alloy coiled into a 0.33 mm (inner diameter) structure.

In various embodiments, the length l₂ of the conductor 714 is in a range of 1 to 50 mm and more preferably, 1 to 20 mm. In one embodiment, the length l₂ of the conductor 714 is 10 mm. In various embodiments, the length l₃ from the tip of the proximal end of the connector system 707 to the tip of the distal end of the second electrode 715 is in a range of 460 to 580 mm. In one embodiment, the length l₃ from the tip of the proximal end of the connector system 707 to the tip of the distal end of the second electrode 715 is 521 mm. Extending distally from the distal end of the second electrode 715 is a length of suture 716 used for guiding and/or securing the lead 700 during implantation. In various embodiments, the length l₄ of the suture 716 is in a range of 1 to 50 mm and more preferably, 1 to 20 mm. In one embodiment, the length l₄ of the suture 716 is 9 mm. In one embodiment, the suture 716 is comprised of nylon.

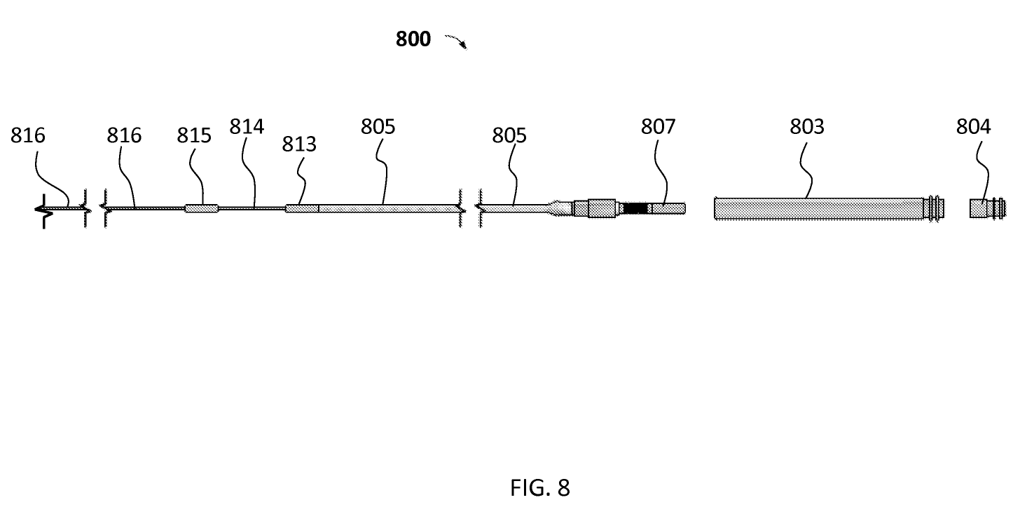

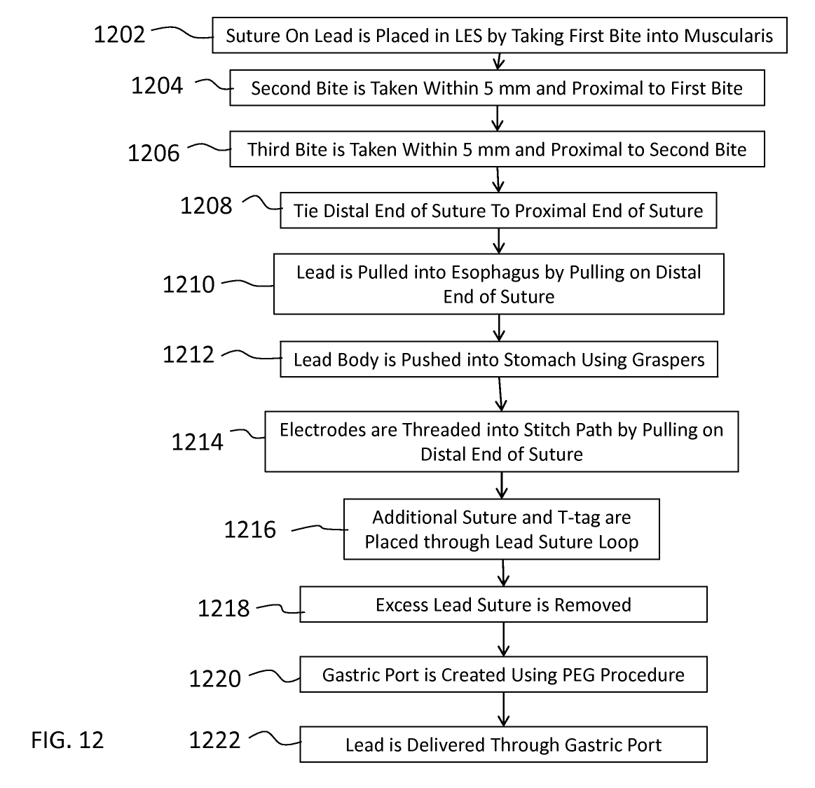

Referring to FIG. 7A, in one embodiment, a loop 717 is formed at the distal end of the length of suture 716. In another embodiment (for example, seen in FIGS. 7B and 8), the distal end of the lead ends in a free end of the length of suture 716. The loop 717 is secured by a knot 718 and a suture tail 719 extends from the knot 718. In various embodiments, the diameter d₁ of the loop 717 at its widest point is in a range of 1 to 20 mm and more preferably, 1 to 10 mm. In one embodiment, the width d₁ of the loop 717 at its widest point is 5 mm. In various embodiments, the suture tail has a length in a range of 100 to 500 mm. In one embodiment, the suture tail has a length of 300 mm. In one embodiment, the loop 717, knot 718, and suture tail 719 are comprised of nylon. The lead 700 is designed to be implanted through the working channel of an endoscope. A physician inserts an endoscope into a patient using natural orifice transluminal endoscopic surgery (NOTES), as described above. For the present embodiment, once the distal end of the endoscope is positioned proximate the target anatomy, the physician uses endoscopic graspers to grasp the loop 717 of the lead 700 and then pulls the lead 700 through the working channel of the endoscope. The suture tail 719 is provided to give the physician another object to grab and pull with other than the loop 717 itself. In one embodiment, the tail 719 also serves to help ‘thread’ the lead 700 into a percutaneous endoscopic gastrostomy (PEG) port (as described with reference to FIG. 12 below) and lead the way to pull the lead into the proper position in vivo. Alternatively, in various embodiments, the lead could be passed through a working channel of a laparoscopic and pulled through the endoscopic tunnel proximate to the target tissue thus eliminating the need to dissect to expose the target tissue. The electrodes 713, 715 are then positioned proximate the target anatomy. In one embodiment, the lead 700 is then anchored in position by suturing through the loop 717 at the distal end of the lead 700. In one embodiment, the loop 717 size is set during manufacturing and the knot 718 is fixed. As the lead 700 is manufactured, the loop 717 size can be made larger or smaller depending upon the intended application. In another embodiment, the loop 717 size is adjustable and the knot 718 is not fixed. The loop 717 size can be adjusted by holding the knot 718 securely and pulling on the length of suture 716, loop 717 itself, or tail 719 to increase or decrease the loop 717 size.

FIG. 8 is an exploded side view illustration of one embodiment of an in-line bipolar implantable electrical stimulation lead 800. Extending distally from a proximal end, FIG. 8 depicts a connector retention ring 804, connector sleeve 803, connector system 807, lead body 805, first electrode 813, conducting wire 814, second electrode 815, and length of suture 816.

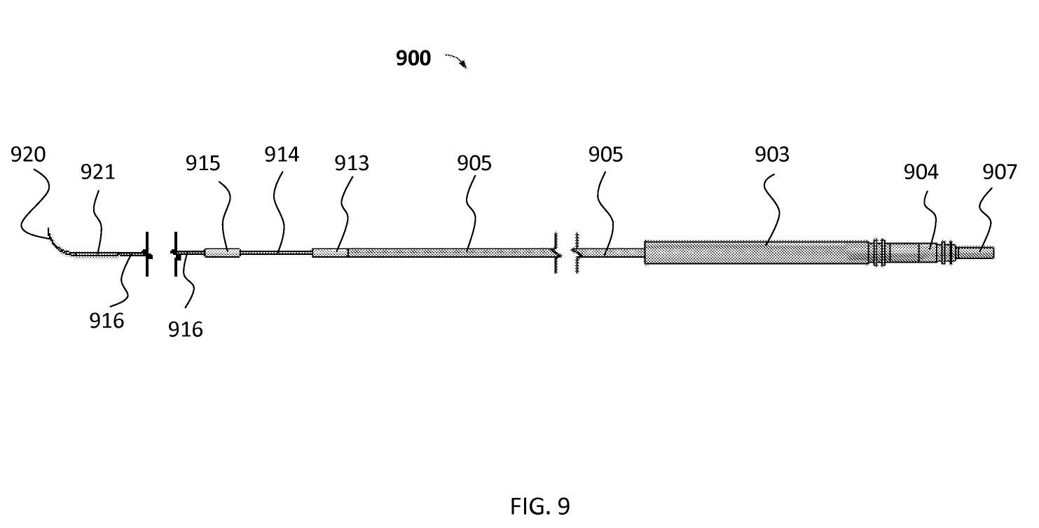

FIG. 9 is a side view illustration of another embodiment of an in-line bipolar implantable electrical stimulation lead 900. Referring to FIG. 9, the lead 900 depicted is similar to the lead 700 depicted in FIG. 7 in that it includes a connector system 907, a connector retention ring 904, a connector sleeve 903, a flexible, elongate lead body 905, a first electrode, a conducting wire 914, a second electrode 915, and a length of suture 916. Rather than a loop or free end, attached to the distal end of the length of suture 916 is a suture needle 920. In various embodiments, the suture needle 920 is similar to the needle 500 described in FIG. 5. The lead is designed to be implanted using a standard laparoscopic technique common in the prior art and can also be implanted using the other various techniques described in the present specification. In various embodiments, the lengths of the various components of the lead 900 are similar to those lengths described for lead 700 of FIG. 7A. The lead body length is greater than that encountered in the prior art, which often measures approximately 350 mm. The greater length allows for greater variation in implantation site. A physician can implant the lead from a more cosmetically pleasing position, for example, a sub-bikini line implantation site or a transumbilical implantation site. The resulting stimulator implant scar would not be visible on the patient's abdomen. In addition, the greater length allows for appropriate routing of the lead to prevent entanglement in the small bowel or a gravid uterus in a female with child bearing potential.

Although reference is made in the figures above to in-line leads having two electrodes, embodiments are envisioned of in-line leads having more than two electrodes. For example, in various embodiments, a unibody, in-line multi-electrode lead includes 4, 6, 8, 10, 12, 14, 16, or more electrodes. In one embodiment, wherein an in-line multi-electrode lead comprises 16 electrodes, each electrode has a length of 1 mm and the lead includes a 1 mm length of conductor (tightest spacing) between each electrode. In other embodiments of unibody leads having more than two electrodes, the leads further include multiple lengths of conductors between the electrodes. In various embodiments, the conductors all have the same length, therefore spacing each electrode the same distance apart from one another. In other embodiments, the conductors all have different lengths, therefore spacing each electrode a different distance apart from one another. In yet other embodiments, some conductors have the same length while others have different lengths, therefore spacing some electrodes the same distance apart and other electrodes different distances apart from one another. These multi-electrode configurations provide the physician with a plurality of options for where and how to stimulate.

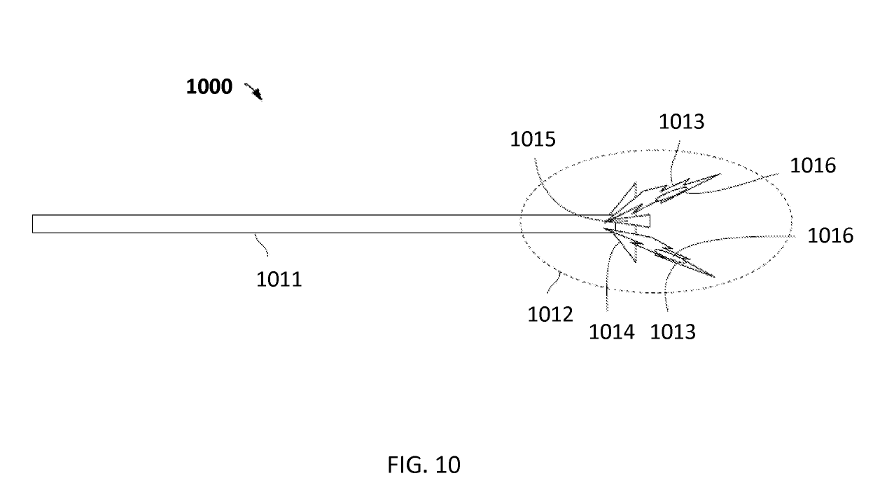

FIG. 10 is a side view illustration of one embodiment of a lead delivery catheter 1000 used to implant the needleless electrical stimulation lead described above using the natural orifice transluminal endoscopic surgery (NOTES) technique. The catheter 1000 includes a catheter body 1011 having a proximal end, a distal end, and a lumen within. In one embodiment, the catheter 1000 has an inflatable balloon 1012 attached to its distal end. The inflatable balloon 1012 is used to perform blunt dissection during implantation. The catheter 1000 also includes a grasping mechanism 1013 at its distal end for grasping the lead. In one embodiment, the grasping mechanism 1013 comprises a pair of opposing grasping members having teeth for grasping the suture loop of the lead. In one embodiment, the catheter 1000 also includes a light source 1014 at its distal end for illumination of the implantation area. The light source 1014 illuminates the implantation tunnel created using the catheter 1000. In one embodiment, the catheter 1000 further includes a camera 1015 at its distal end for visualization of the implantation area. The light source 1014 illuminates the tunnel so that it can be visualized using the camera 1015. In one embodiment, the catheter 1000 further includes a bipolar electrode 1016 for electrocautery of tissues as the implantation site. In one embodiment, the bipolar electrode 1016 is incorporated into the grasping mechanism 1013. The bipolar electrode 1016 is used to create a primary incision, for dissection in the implantation tunnel, and/or for hemostasis during the implantation procedure.

The lead delivery catheter 1000 can be used to implant one or more leads via the NOTES technique using an endoscopic approach or a laparoscopic approach. For example, when placing leads proximate the lower esophageal sphincter (LES), an incision is made with the catheter tip in the esophageal wall at least one inch proximal to the LES using an endoscopic approach. Using a laparoscopic approach, an incision is made with the catheter tip in the gastric wall at least one inch distal to the LES. In both approaches, the distal end of the catheter is then advanced through the incision. Air is then pumped through the catheter lumen to inflate the balloon attached to the distal end of the catheter. The inflated balloon is used to create a submucosal or subserosal pocket using blunt dissection. The distal end of the catheter is then further advanced into the pocket and the balloon is deflated and re-inflated to extend the pocket longitudinally, creating a tunnel for the passage of the lead.

In the endoscopic approach, once an adequate tunnel has been created that crosses the implant site, a second incision is made on the contralateral side to create an exit through the gastrointestinal wall. A laparoscopic trocar is inserted into the abdomen with its distal end passing through the second incision. The catheter is advanced further and the lead is passed through the laparoscopic trocar, grasped by the grasping mechanism, and pulled into the created tunnel. The lead is then positioned proximate the LES. In the endoscopic approach, the lead can also be passed through an abdominal incision directly and grasped using the grasping mechanism of the catheter. The lead and the endoscope with the catheter are withdrawn into the tunnel and the lead is released once the electrodes are in the desired postion proximate to the LES muscles. In the laparoscopic approach, once an adequate tunnel has been created that crosses the implant site, the catheter is removed from the endoscope. The lead is then passed through a working channel of the endoscope. The catheter is reinserted through a laparoscopic trocar and advanced to the implant site. Using the grasping mechanism, the physician grabs the lead which is then positioned proximate the LES. Over time, fibrosis about the anchors permanently fixes the lead in the tunnel with the stimulating electrodes proximate the LES. In one embodiment, temporary sutures or clips are used to provide temporary anchoring support while fibrosis is setting in about the anchors. The temporary sutures or clips are later removed after permanent anchoring has been achieved with the lead anchors.

Optionally, in another embodiment, the lead is delivered to the implantation site using a laparoscopic method with tunneling from the outside inwards. This implantation is performed completely laparoscopically without the need for an opening at the distal end of the implantation tunnel. The physician laparoscopically creates a dead-end tunnel proximate the target tissues. The lead is then pushed into the blind tunnel and allowed to anchor over time.

Optionally, in another embodiment, the lead is delivered to the implantation site via a completely endoscopic procedure. Using an endoscope and the lead delivery catheter, the physician creates a tunnel as described above. The lead is passed through the endoscope and placed into position using the grasping mechanism of the catheter.

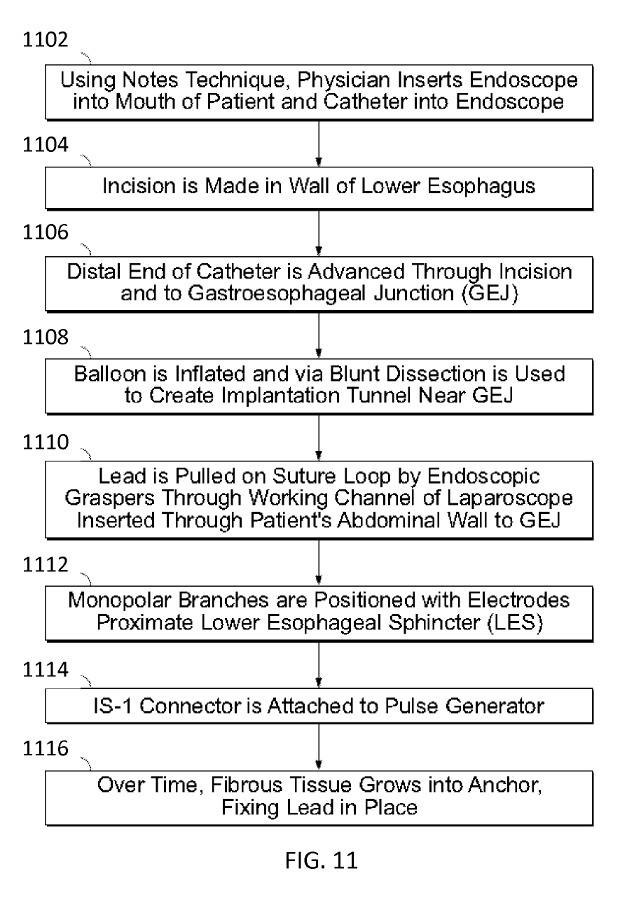

FIG. 11 is a flowchart illustrating one embodiment of the steps involved in implanting a needleless electrical stimulation lead using an endoscope. The lead is of the type having the suture material loop and anchors as described with reference to FIGS. 6 above. At step 1102, using the NOTES technique, a physician inserts an endoscope into the mouth of a patient with lower esophageal sphincter (LES) dysfunction. A lead delivery catheter as described with reference to FIG. 10 is also inserted into a working channel of the endoscope. At step 1104, an incision is made in the wall of the lower esophagus. The distal end of the catheter is then advanced through the incision and into an area proximate the GEJ at step 1106. At step 1108, the balloon at the distal end of the catheter is inflated and used to create an implantation tunnel using blunt dissection. Then, at step 1110, the lead is pulled by endoscopic graspers through a laparoscope that has been inserted into the patient's abdomen to the tunnel created proximate the GEJ. The monopolar branches, or single branch, when using the in-line lead, of the lead are/is then positioned with the electrodes proximate the LES at step 1112. At step 1114, the IS-1 connector at the other end of the lead is attached to a pulse generator. Over time, at step 1116, fibrous tissue grows into the anchor, fixing the lead in place.

FIG. 12 is a flowchart illustrating one embodiment of the steps involved in endoscopically implanting an in-line bipolar electrical stimulation lead similar to the lead described with reference to FIGS. 7A, 7B, and 8. At step 1202, a suture is placed in a lower esophageal sphincter (LES) by taking a first bite, from the bottom up, deep enough to reach the muscularis. At step 1204, a second bite is taken, in similar fashion, 5 mm or closer, and proximal to, the first bite. A third bite is taken, in similar fashion, 5 mm or closer, and proximal to, the second bite, at step 1206. Then, at step 1208, the distal end of the suture is tied to the proximal end of the suture extending from the LES. The distal end of the suture is then pulled upward to pull the lead into the esophagus at step 1210, under vision, until the electrodes are near the entry point of the stitch path made by the suture. Then, at step 1212, using graspers, the entire lead body is pushed into the stomach. The remaining suture is pulled upward at step 1214 to thread the electrodes through the stitch path, making sure the electrodes are buried (no longer visible in the esophageal lumen). At step 1216, an additional suture and t-tag are placed through the suture loop of the lead to ensure solid anchoring of the lead and prevent migration. Excess suture from the lead is cut and removed at step 1218. A gastric port is created at step 1220 using a percutaneous endoscopic gastrostomy (PEG) procedure. Finally, at step 1222, the lead is delivered through the gastric port. Optionally, in one embodiment, the lead includes a suture loop at its distal end and the lead is pulled in the steps above via the suture loop.

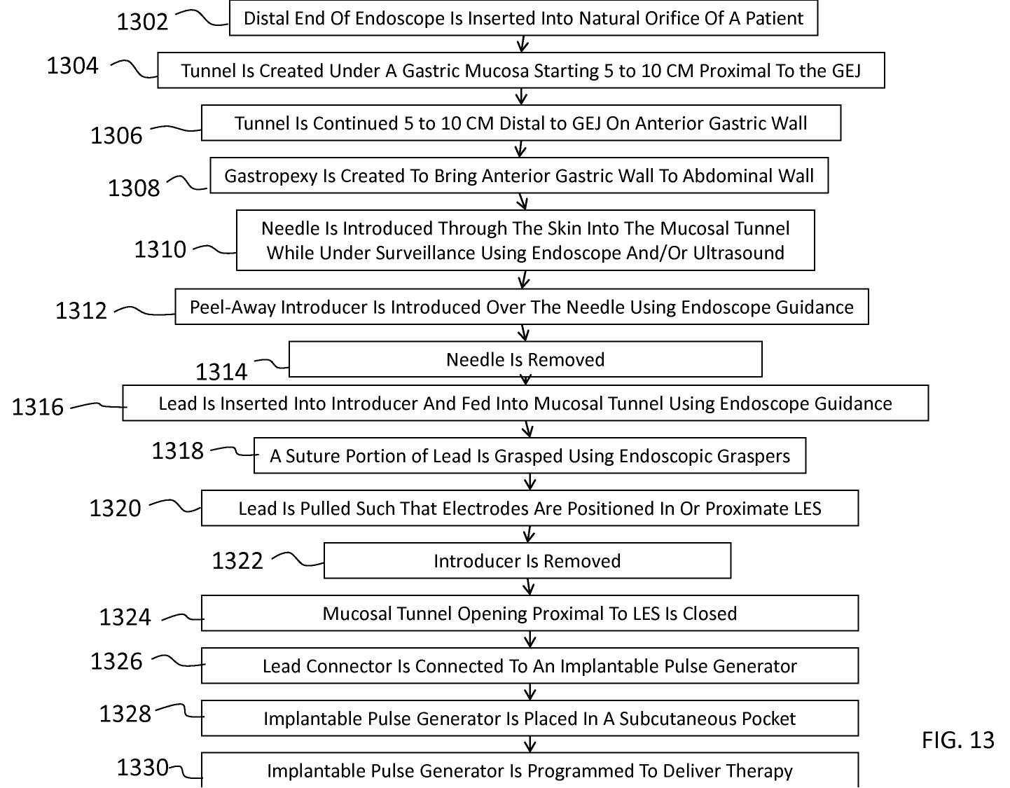

FIG. 13 is a flowchart illustrating one embodiment of the steps involved in a method of implanting an electrical stimulation lead having a connector and a plurality of in-line electrodes into a patient. At step 1302, the distal end of an endoscope is inserted into a natural orifice of a patient. A tunnel is created under the gastric mucosa starting 5 cm to 10 cm proximal to the gastroesophageal junction (GEJ) at step 1304. Tunneling is continued 5 cm to 10 cm distal to the GEJ on an anterior gastric wall at step 1306. Then, at step 1308, a gastropexy is created to bring the anterior gastric wall to an abdominal wall. Gastropexy is a surgical operation in which the stomach is sutured to the abdominal wall or the diaphragm.

At step 1310, a needle is introduced through the skin into the mucosal tunnel while under surveillance using the endoscope and/or ultrasound to guide the needle to the correct location. A peel-away introducer is introduced over the needle into the mucosal tunnel under guidance from the endoscope at step 1312. At step 1314, the needle is removed. The electrical stimulation lead is inserted into the introducer and fed into the mucosal tunnel under guidance from the endoscope at step 1316. Then, at step 1318, a suture portion of the electrical stimulation lead is grasped using endoscopic graspers. The electrical stimulation lead is then pulled at step 1320 such that the electrodes are positioned in or proximate a lower esophageal sphincter (LES). The introducer is removed at step 1322. An opening of the mucosal tunnel proximal to the LES is closed at step 1324. The electrical stimulation lead connector is connected to an implantable pulse generator at step 1326. At step 1328, the implantable pulse generator is placed in a subcutaneous pocket. Finally, at step 1330, the implantable pulse generator is programmed to deliver therapy.

In various embodiments, the method described above optionally includes the step of anchoring the electrical stimulation lead to the muscularis of the LES. In one embodiment, the lead is anchored to the muscularis of the LES by any conventional suturing mechanism. In another embodiment, the lead is anchored to the muscularis of the LES by using sutures which contain micro-barb structures. In another embodiment, the lead is anchored to the muscularis of the LES by employing a barb-like element which anchors itself when the lead is pulled. In yet another embodiment, the lead is anchored to the muscularis of the LES by use of a biomaterial which promotes tissue in-growth including any one or combination of porous silicone and tissue scaffolds.

The above examples are merely illustrative of the many applications of the system of the present invention. Although only a few embodiments of the present invention have been described herein, it should be understood that the present invention might be embodied in many other specific forms without departing from the spirit or scope of the invention. Therefore, the present examples and embodiments are to be considered as illustrative and not restrictive, and the invention may be modified within the scope of the appended claims.

We claim:

1. An in-line implantable electrical lead for use in the stimulation of biological tissues, said lead comprising:
an insulated, flexible, elongate lead body having a proximal end and a distal end;
a connector attached to and in electrical communication with said proximal end of said lead body;
a plurality of electrodes comprising at least a most proximal electrode and a most distal electrode, said electrodes being arranged in-line and spaced a predetermined distance apart from one another, wherein said most proximal electrode is attached to said distal end of said lead body;
at least one conductor positioned between and extending through each of said plurality of electrodes, thereby connecting each of said plurality of electrodes; and,
a suture extending distally from said most distal electrode;
wherein a first length extending from a tip of a proximal end of said connector to a tip of a distal end of said most proximal electrode is in a range of 450 to 550 mm and a second length of said conductor is in a range of 1 to 50 mm.

2. The implantable electrical lead of claim 1, wherein said plurality of electrodes is equal to two.

3. The implantable electrical lead of claim 1, wherein said plurality of electrodes is equal to four.

4. The implantable electrical lead of claim 1, wherein said plurality of electrodes is equal to eight.

5. The implantable electrical lead of claim 1, wherein each of said plurality of electrodes has a length in a range of 1 to 25 mm and a width in a range of 0.10 to 1.50 mm.

6. The implantable electrical lead of claim 1, wherein said lead body is comprised of a plurality of coils or cables.

7. The implantable electrical lead of claim 1, wherein a width of said lead body is in a range of 0.20 to 2.00 mm.

8. The implantable electrical lead of claim 1, wherein said conductor is comprised of a plurality of conductors.

9. The implantable electrical lead of claim 1, wherein said lead comprises more than two electrodes and two or more conductors.

10. The implantable electrical lead of claim 9, wherein each conductor has the same or different lengths or some conductors have the same length while other conductors have different lengths.

11. The implantable electrical lead of claim 1, further comprising a suture loop and a suture tail formed from said suture extending distally from said second electrode.

12. The implantable electrical lead of claim 11, wherein a diameter of said suture loop about its widest point is in a range of 1 to 20 mm.

13. The implantable electrical lead of claim 11, wherein a third length extending from a distal end of said second electrode to a knot forming said loop is in a range of 1 to 20 mm and a fourth length of said suture tail is in a range of 100 to 500 mm.

14. The implantable electrical lead of claim 11, wherein said diameter of said suture loop is fixed.

15. The implantable electrical lead of claim 11, wherein said diameter of said suture loop is adjustable by pulling on a portion of said suture, suture loop, or suture tail.

16. The implantable electrical lead of claim 1, further comprising a needle attached to a distal end of said suture.

17. The implantable electrical lead of claim 16, wherein said needle is within a range of a ¼ to ⅜ of a circle curve needle with a length ranging from 13 to 28 mm and includes a base having a diameter in a range of 0.58 mm to 0.88 mm.

18. The implantable electrical lead of claim 16, wherein said needle comprises a straight proximal portion having a first length within a range of 8 mm to 16 mm, a curved distal portion having a second length within a range of 4 mm to 10 mm, and an opening at a proximal end of said straight proximal portion configured to fixedly receive a length of suture and extending at least 1.6 mm within said straight proximal portion, further wherein a tapered point at a distal end of said curved distal portion is offset from an axis of said straight proximal portion by a distance within a range of 1 mm to 5 mm.

19. The implantable electrical lead of claim 1, further comprising a sleeve covering a proximal portion of said lead body and a distal portion of said connector.

20. The implantable electrical lead of claim 19, further comprising a retention ring positioned proximal to said sleeve and securing said sleeve in place.

21. An in-line implantable electrical lead for use in the stimulation of biological tissues, said lead comprising:
an insulated, flexible, elongate lead body having a proximal end and a distal end;
a connector attached to and in electrical communication with said proximal end of said lead body;
a first electrode attached to said distal end of said lead body;
a second electrode attached to said first electrode by a connecting conducting cable, said second electrode being in-line with and spaced distally apart from said first electrode; and,
a suture extending distally from said second electrode;
wherein a first length extending from a proximal end of said connector to a distal end of said first electrode is in a range of 450 to 550 mm and a second length of said connecting conducting cable is in a range of 1 to 50 mm.

22. A method of endoscopically implanting an electrical stimulation lead having a connector, a lead body, a first electrode, a second electrode in-line with said first electrode, and a suture extending distally from said second electrode, said method comprising the steps of:
stitching said suture at least once through the muscularis of a lower esophageal sphincter (LES);
tying a distal end of said suture to a proximal end of said suture;
pulling on a distal end of said suture to pull said lead body into an esophagus;
pushing said lead body into a stomach using graspers;
pulling on said distal end of said suture to thread electrodes into stitch path;
suturing at least one additional suture and T-tag through a suture loop created with said suture of said lead;
removing excess suture from said lead;
creating a gastric port using a percutaneous endoscopic gastrostomy (PEG) procedure; and,
delivering said lead through said gastric port.

23. The method of claim 22, wherein said lead further includes a loop formed from said suture and said steps of pulling on said distal end of said suture comprise pulling on said loop.

24. A method of implanting an electrical stimulation lead having a connector and a plurality of in-line electrodes into a patient, said method comprising the steps of:
inserting the distal end of an endoscope into a natural orifice of a patient;
creating a tunnel under a gastric mucosa, wherein said tunnel begins 5 cm to 10 cm proximal to the gastroesophageal junction (GEJ);
continuing said tunnel 5 cm to 10 cm distal to the GEJ on an anterior gastric wall;
creating a gastropexy to bring the anterior gastric wall to an abdominal wall;
introducing a needle through the skin into the mucosal tunnel while under surveillance using the endoscope and/or ultrasound to guide the needle to the correct location;
introducing a peel-away introducer over the needle into the mucosal tunnel under guidance from the endoscope;
removing said needle;
inserting the electrical stimulation lead into the introducer and feeding said lead into the mucosal tunnel under guidance from the endoscope;
grasping a suture portion of the electrical stimulation lead using endoscopic graspers;
pulling the electrical stimulation lead such that the electrodes are positioned in or proximate a lower esophageal sphincter (LES);
removing said introducer;
closing an opening of the mucosal tunnel proximal to the LES;
connecting the electrical stimulation lead connector to an implantable pulse generator;
placing said implantable pulse generator in a subcutaneous pocket; and
programming said implantable pulse generator to deliver therapy.

25. The method of claim 24, further comprising the step of anchoring said electrical stimulation lead to a muscularis of the LES.

26. The method of claim 25, wherein said anchoring is achieved by any one or combination of a conventional suturing mechanism, using sutures which contain micro-barb structure, employing a barb-like element which anchors itself when said lead is pulled, and use of a biomaterial which promotes tissue in-growth, including any one or combination of porous silicone and tissue scaffolds.
